(19) United States
(12) Patent Application Publication
(10) Pub. No.: US 2020/0171266 A1
(43) Pub. Date: Jun. 4, 2020

(54) AROMATHERAPY VAPORIZATION DEVICE

(71) Applicant: Michael Alexander Trzecieski, Toronto (CA)

(72) Inventor: Michael Alexander Trzecieski, Toronto (CA)

(21) Appl. No.: 16/781,669

(22) Filed: Feb. 4, 2020

Related U.S. Application Data
(63) Continuation-in-part of application No. 14/614,005, filed on Feb. 4, 2015, now abandoned.
Application No. 14/614,005 is a continuation-in-part of application No. 14/829,660, filed on Aug. 19, 2015, now Pat. No. 10,238,764.
Application No. 14/829,660 is a continuation-in-part of application No. 16/009,259, filed on Jun. 15, 2018.
(60) Provisional application No. 61/935,349, filed on Feb. 4, 2014.
(60) Provisional application No. 62/038,863, filed on Aug. 19, 2014.
(60) Provisional application No. 62/800,956, filed on Feb. 4, 2019.

(51) Int. Cl. A61M 21/00; A61M 11/04

(52) U.S. Cl. CPC A61M 21/00; A61M 11/042; A61M 15/06; A61M 2205/3368; A61M 2021/0016; A61M 2205/8206

(57) ABSTRACT

A vaporization device includes a housing, a fluid pathway extending through the housing with a heating element proximate a housing first end, a control circuit and a battery disposed within the housing. The heating chamber is in fluid communication with the fluid pathway. The heating element is operable to generate heat at a predetermined vaporization temperature. The control circuit has a switch operable to control a flow of current from the battery to the heating element. In use, the predetermined vaporization temperature is sufficient to vaporize phyto material in contact with the heat at the predetermined vaporization temperature to emit a vapor. Inhaling from the housing second end draws mixed vapor and air through the fluid pathway. A removable container may be used for containing of the phyto material extract being contacted by the heating element and the removable container may be releasably coupled with the housing where the heating element may be inserted into the removable storage container for heating of the phyto material for creating a vapor therefrom.

### CROSS REFERENCE TO RELATED APPLICATIONS

This application is a continuation in part of U.S. patent application Ser. No. 14/614,005, filed on Feb. 4, 2015, which claims the benefit of U.S. Provisional Patent Application No. 61/935,349 filed on Feb. 4, 2014, and U.S. patent application Ser. No. 14/829,660 filed on Aug. 19, 2015, which claims the benefit of U.S. Provisional Patent Application No. 62/038,863 filed on Aug. 19, 2014, and also claims priority from U.S. Provisional Patent Application No. 62/519,972, filed Jun. 15, 2017 and this application is a continuation in part of U.S. patent application Ser. No. 16/009,259 and claims the benefit of United States Provisional Patent Application, 2 filed on Feb. 4, 2019, the entire contents of each of which are hereby incorporated by reference.

### FIELD OF THE INVENTION

This disclosure relates generally to vaporization devices, and in particular to vaporization devices that vaporize phyto material or phyto material extracts for use in aromatherapy.

### BACKGROUND

The following is intended to introduce the reader to the detailed description that follows and not to define or limit the claimed subject matter.

Aromatherapy generally uses essential oils released from phyto materials, such as the leaves of plants, for therapeutic benefits. By heating phyto material to a temperature sufficient for vaporization, essential oils and extracts may be emitted from the phyto material as vapor. This vapor may be inhaled by a user for its therapeutic benefits. Different phyto materials release vapors at different temperatures. For example, some phyto materials release vapor at 120 degrees Celsius, while others release vapor at 220 degrees Celsius. Some phyto material extracts release vapor at over 500 degrees Fahrenheit to 700 degrees Fahrenheit. Devices that may heat phyto material to a temperature sufficient to release the vapor are generally known as vaporizers. Various devices for vaporizing phyto materials for aromatherapy are known.

### SUMMARY

The following introduction is provided to introduce the reader to the more detailed description to follow and not to limit or define any claimed or as yet unclaimed invention. One or more inventions may reside in any combination or sub-combination of the elements or process steps disclosed in any part of this document including its claims and figures.

In accordance with a first aspect of this disclosure, there is provided a vaporization device. The vaporization may include a housing extending from a first end to a second end; a heating chamber disposed at the first end of the housing, the heating chamber having a first chamber end proximate the first end of the housing, a second chamber end displaced from the first end towards the second end and a chamber wall extending between the first chamber end and the second chamber end, wherein a volume bounded by the first chamber end, the second chamber end and the chamber wall defines a phyto material receiving chamber, and wherein the first chamber end is open and the second chamber end is substantially closed and comprises one or more vents; an inhalation aperture formed proximate the second end of the housing; a heating element disposed within the heating chamber between the first chamber end and the second chamber end, the heating element configured to emit heat, wherein phyto material is receivable in the phyto material receiving chamber between the heating element and the chamber wall whereby heat emitted from the heating element is operable to at least partially vaporize the phyto material whereby a vapor is emitted; an energy storage member at least partially disposed within the housing; a control circuit electrically coupled to the energy storage member and to the heating element, the control circuit configured to control the flow of electrical current from the energy storage member to the heating element, the control circuit further comprising a user-activated switch for controlling the flow of electrical current from the energy storage member to the heating element; a first fluid pathway extending through the housing from the first end to the second end, the fluid pathway being fluidly coupled to the phyto material receiving chamber and to the inhalation aperture; an ambient air input port; and a second fluid pathway extending between the ambient air input port and a second fluid pathway end disposed proximate the second chamber end, the second fluid pathway end fluidly coupled to the inhalation aperture, the second fluid pathway comprising a puff sensor coupled to the control circuit, the puff sensor operable to detect the flow of ambient air through the second fluid pathway and to generate an airflow signal in response to detecting the flow of ambient air, wherein the control circuit is operable to control the flow of electrical current from the energy storage member to the heating element in response to the airflow signal; wherein, upon activation of the switch, electrical current from the energy storage member is enabled to be provided to the heating element; and upon creation of a low pressure at the inhalation aperture, a reduced pressure region is created in the first fluid pathway and ambient air is induced to flow into the heating chamber via the first end and propagate through the vapor emitted from heating the phyto material, whereby the vapor mixes with the ambient air and together flows through the fluid pathway to the inhalation aperture, and ambient air separately enters the ambient air input port whereby the puff sensor is triggered.

In some embodiments, the heating element may be a blade-shaped heating element.

In some embodiments, the blade-shaped heating element may include two parallel flat long sides and two parallel flat narrow sides and a majority of the heat from the heating element radiates from the two parallel flat long sides outwardly towards the chamber wall.

In some embodiments, the heating chamber may be cylindrical and the first chamber end may have a sharpened peripheral edge.

In some embodiments, the heating element may be a rod-shaped heating element.

In some embodiments, the first fluid pathway may include a thermally conductive material proximate the inhalation aperture to provide heat transfer between ambient air and the mixed vapor and air flowing through the fluid pathway.

In some embodiments, the closed end may include a sliding member that is movable between a first position in which the sliding member is proximate to the second chamber end and second position in which the sliding member is proximate to the first chamber end.

In some embodiments, the user-activated switch may be a mechanical switch.

In accordance with an aspect of the disclosure, there is provided a vaporization device. The vaporization device includes a housing extending axially between a housing first end and a housing second end; a fluid pathway extending through the housing between the housing first end and the housing second end; an inhalation aperture fluidly coupled to the fluid pathway, the inhalation aperture positioned at the housing second end; a heating element heatable to at least one predetermined vaporization temperature, wherein in use the predetermined vaporization temperature is selected to vaporize phyto material proximate the heating element whereby a phyto material vapor is emitted; an energy storage member disposed within the housing and electrically coupled to the heating element; and a control circuit disposed within the housing and electrically coupled to the energy storage member, the control circuit having a user-activated switch operable to control a flow of electric current from the energy storage member to the heating element, wherein, in response to a user inhalation at the inhalation aperture, a pressure gradient is created across the fluid pathway that draws ambient air from the external environment into the fluid pathway and the ambient air mixes with the phyto material vapor, and the mixed vapor and air are drawn through the fluid pathway to the inhalation aperture.

In some embodiments, the vaporization device may include a heating chamber proximate the housing first end, the heating chamber having a first heating chamber end, a second heating chamber end in fluid communication with the fluid pathway, and a chamber wall extending between the first heating chamber end and the second heating chamber end, the first heating chamber end, second heating chamber end and chamber wall together defining a heating chamber volume, wherein the first heating chamber end is open and the second heating chamber end is a substantially closed end comprising one or more vents.

In some embodiments, an inner portion of the fluid pathway may include a thermally conductive liner.

In some embodiments, the heating chamber may include a ceramic material.

In some embodiments, the user-activated switch may be a mechanical switch.

In some embodiments, the fluid pathway may include an airflow sensor electronically coupled to the control circuit, and the control circuit may be configured to determine a volume of ambient air entering the heating chamber based on airflow readings received from the airflow sensor and provide an indication that the phyto material in the internal cavity needs replacing when the determined volume of ambient air entering the heating chamber exceeds a predetermined volume threshold.

In some embodiments, the vaporization device may include a secondary fluid pathway extending between an ambient air input port and a secondary inhalation aperture, where the secondary inhalation aperture and the inhalation aperture are adjacent one another and are formed as a joint inhalation aperture at the housing second end, the secondary fluid pathway having a puff sensor therein configured to detect a flow rate within the secondary fluid pathway, where a primary pressure gradient is created across the fluid pathway and a secondary pressure gradient is created across the secondary fluid pathway in response to the user inhalation, the primary pressure gradient drawing a first volume of ambient air from the external environment into the heating chamber volume and the secondary pressure gradient drawing a second volume of ambient air from the external environmental into the air input port, and the second volume of ambient air triggers the puff sensor to detect the flow rate of the second volume of ambient air and send a puff signal to the control circuit to adjust the flow of the electric current from the energy storage member to the heating element based on the detected flow rate.

In some embodiments, a central axis of the housing may be offset from a central axis of the fluid pathway.

In some embodiments, the vaporization device may include a first contact and a second contact, the first contact and the second contact being electrically coupled to the control circuit and protruding from the housing, where the first contact and the second contact are respectively engageable with a first energy storage member recharging contact and a second energy storage member recharging contact of a recharging hub to provide electrical energy from the recharging port to the energy storage member.

In some embodiments, the vaporization device may include a heating chamber that defines a phyto material receiving area and the heating element may be positioned within the phyto material receiving area.

In some embodiments, the heating chamber may be cylindrical and the outer end of the heating chamber may have a pointed peripheral edge operable to cut phyto material when pressed against it.

In some embodiments, the heating element may include a blade aligned centrally within an internal cavity of the heating chamber, the blade may be configured to radiate heat outwardly.

In some embodiments, the heating element may be a rod-shaped heating element aligned centrally within an internal cavity of the heating chamber, the rod-shaped heating element may be configured to radiate heat outwardly

In some embodiments, the heating element may contact a chamber wall of the heating chamber and may be configured to heat the chamber wall.

In some embodiments, the heating chamber extends between an outer heating chamber end and an inner heating chamber end in fluid communication with the fluid pathway, where the inner heating chamber end is a substantially closed end comprising one or more vents; the closed end includes a sliding member that is slideable between an open position and a closed position, in the opened position, the sliding member is spaced apart from the outer heating chamber end such that the phyto receiving area is accessible to receive phyto material; in the closed position, the sliding barrier is positioned proximate to the outer heating chamber end.

In some embodiments, the heating element includes a protruding portion that protrudes from the housing first end.

In some embodiments, the heating element may be a flat plate ceramic heating element.

In some embodiments, the device may include a cylindrical heating chamber that extends along a central axis; and the flat plate ceramic heating element may be offset from the central axis.

In some embodiments, the heating element may be a cylindrical heating element.

In accordance with an aspect of the disclosure, there is provided a vaporization device. The vaporization device may include a vaporization tube extending axially between an open first tube end and an open second tube end, the vaporization tube having an outer tube surface enclosing a fluid pathway extending between the first tube end and the second tube end, wherein the second tube end defining an inhalation aperture; a cylindrical heating element coupled to the outer tube surface proximate the open first tube end, the heating element being concentrically aligned with the vaporization tube, wherein the cylindrical heating element is operable to heat the fluid pathway at the open first tube end to a predetermined vaporization temperature, wherein in use the predetermined vaporization temperature is sufficient to vaporize phyto material in contact with the open first tube end; an energy storage member electrically coupled to the cylindrical heating element; and a control circuit electrically coupled to the energy storage member, the control circuit operable to control a flow of electric current from the energy storage member to the heating element, wherein, in response to a user inhalation at the inhalation aperture, a pressure gradient is created across the fluid pathway that draws ambient air from the external environment into the open tube first end and the ambient air mixes with the phyto material vapor, and the mixed vapor and air are drawn through the fluid pathway to the inhalation aperture.

In some embodiments, the vaporization tube may be a glass tube.

In some embodiments, the vaporization tube may include one of a fused quartz glass and a borosilicate glass tube.

In accordance with an aspect of the disclosure, there is provided a vaporization device comprising: a housing having a longitudinal axis and a housing first end and a housing second end opposite the housing first end; a slider assembly for sliding parallel with the longitudinal axis and in relation with the housing; a vapor conduit extending axially between a first open conduit end and a second open conduit end, the vapor conduit having an outer conduit surface enclosing a fluid pathway extending between the first open conduit end and the second open conduit end, wherein the second open conduit end defining an inhalation aperture; a removable storage container bounded by at least two sidewalls and having an open end forming a storage cavity for receiving and for storing of phyto material extract, the removable storage container open end for engaging with the housing first end for forming a substantially enclosed heating chamber when the storage container is coupled with the housing; an ambient air input port fluidly coupled with the heating chamber for allowing ambient air to enter into the substantially enclosed heating chamber; a heating element coupled with the slider assembly and proximate the first open conduit end and proximate the housing first end, the heating element operable to heat to a predetermined vaporization temperature wherein in use the predetermined vaporization temperature is sufficient to vaporize phyto material extract in contact with the heating element; an energy storage member electrically coupled to the heating element; and a control circuit electrically coupled to the energy storage member, the control circuit operable to control a flow of electric current from the energy storage member to the heating element, the slider assembly and heating element biased with the heating element other than substantially extended from the housing in a first mode of operation; wherein, in response to a user depressing on the slider assembly the control circuit is engaged for electric current to flow from the energy storage member to the heating element for heating of the heating element to the predefined vaporization temperature and for the heating element sliding away from the first end of the housing towards the phyto material extract and for being at least partially inserted into the storage container in a second mode of operation and the heating element for contacting the phyto material extract and for creating a phyto material extract vapor; and where inhalation at the inhalation aperture, a pressure gradient is created across the fluid pathway that draws ambient air from the ambient air input port from the external environment into the first open conduit end and the ambient air mixes with the phyto material extract vapor and the mixed vapor and air are drawn through the fluid pathway to the inhalation aperture.

In some embodiments a vaporization may include a spring having a spring force disposed between the slider assembly and the housing, wherein the slider assembly is biased by the spring with the force of the spring being overcome to operate from the first mode of operation to the second mode of operation.

In some embodiments a vaporization may include a central plane positioned to contain a line coincident with the longitudinal axis, wherein the fluid pathway first end is offset from the central plane and the heating chamber comprises the open end that is fluidly coupled with the fluid pathway, and the inhalation aperture is also offset from the central plane.

In some embodiments a vaporization may include an energy storage member comprises a rechargeable battery that is electrically coupled to the heating element through the control circuit and the rechargeable battery is coaxial with the central axis.

In some embodiments a vaporization may include a switch electrically coupled with the control circuit, the switch for being coupled with the slider assembly for being activated by the user when depressing on the slider assembly for providing a control signal to the control circuit for electric current to flow from the energy storage member to the heating element.

In accordance with an aspect of the disclosure, there is provided a herbal vaporization device comprising: a housing comprising a first end, a second end disposed opposite the first end disposed coaxially with a slider assembly for sliding within the housing along a coaxial longitudinal axis; a first battery at least partially disposed within the slider assembly; a conductive heating element coupled with the slider assembly and disposed proximate the first end of the housing a pivotable head coupled with the slider assembly proximate the second end of the housing and for extending past the housing second end, the pivotable head for being pivotable about a transverse axis that is approximately perpendicular to the coaxial longitudinal axis with the pivotable head being in a first orientation with an inhalation aperture oriented radially from the coaxial longitudinal axis and in the second orientation the inhalation aperture oriented approximately coaxially with the coaxial longitudinal axis; a switch coupled with the pivotable head opposite the inhalation aperture; a first control circuit coupled with the first battery and the conductive heating element and the switch, the first control circuit for controlling the flow of electrical current from the first battery to the conductive heating element, the switch coupled with the first control circuit for providing a control signal to the first control circuit for affecting the flow of electrical current from the first battery to the conductive heating element for heating of the conductive heating element to the predetermined vaporization temperature; a fluid pathway extending through the slider assembly from the first end of the housing to past the second end of the housing the fluid pathway being fluidly coupled proximate the conductive heating element and terminating at the inhalation aperture; wherein in a first orientation of the pivotable head the conductive heating element is retracted within the housing and upon depressing of the switch, the slider assembly is slid within the housing and the conductive heating element is extended from the housing and heated to the predetermined vaporization temperature in a second orientation of the pivotable head the conductive heating element is extended from the housing and upon depressing of the switch is heated to the predetermined vaporization temperature, the heated conductive heating element for contacting the phyto material extract and where upon creating a low pressure proximate the second end of the fluid pathway, a low pressure is created in the fluid pathway and ambient air at a higher pressure flows and mixes with vapor emitted from conductive heating of the phyto material extract and with the ambient air and together flows out of the inhalation aperture.

In some embodiments a vaporization may include a first housing comprising a central plane positioned to contain a line coincident with the longitudinal axis, wherein the fluid pathway first end is offset from the central plane and the heating chamber comprises the open end that is fluidly coupled with the fluid pathway and the inhalation aperture is also offset from the central plane.

In some embodiments a vaporization may include a removable storage container bounded by at least two sidewalls and having an open end for forming a storage cavity for receiving and for storing of phyto material extract, the removable storage container open end for engaging with the housing first end for forming a substantially enclosed heating chamber when the storage container is coupled with the housing where upon depressing of the switch, the slider assembly is slid within the housing and the conductive heating element is extended from the housing and heated to the predetermined vaporization temperature and contacts the phyto material extract for creating vapor emitted from conductive heating of the phyto material extract.

In accordance with an aspect of the disclosure, there is provided a vaporization device comprising: providing a housing having a longitudinal axis and a housing end and a housing second end opposite the housing first end; providing a slider assembly for sliding the longitudinal axis and in relation with the housing; providing a vapor conduit extending axially between a first open conduit end and a second open conduit end, the vapor conduit having an outer conduit surface enclosing a fluid pathway extending between the first open conduit end and the second open conduit end, wherein the second open conduit end defining an inhalation aperture; providing a heating element coupled with the slider assembly and proximate the housing first end; heating of the heating element to a predetermined vaporization temperature wherein in use the predetermined vaporization temperature is sufficient to vaporize phyto material extract in contact with the heating element for providing phyto material extract vapor; and; extending of the heating element away from the housing first end parallel with the longitudinal axis to contact phyto material extract contained within the storage cavity of the removable storage container; inhaling from the inhalation aperture and creating a pressure gradient across the fluid pathway that draws ambient air from an ambient air input port from the external environment into the first open conduit end and the ambient air mixes with the phyto material extract vapor, and the mixed vapor and air are drawn through the fluid pathway to the inhalation aperture.

In some embodiments a vaporization may include filling a removable storage container bounded by at least two sidewalls and having a storage cavity formed therein for containing phyto material extract; releasably coupling of the removable storage container with the housing so the storage cavity faces the housing, wherein extending of the heating element away from the housing first end parallel with the longitudinal axis comprises contacting phyto material extract contained within the storage cavity of the removable storage container.

In some embodiments a vaporization may include a vaporization device providing a pivotable head coupled with the slider assembly proximate the second end of the housing and for extending past the housing second end, the pivotable head for being pivotable about a transverse axis that is approximately perpendicular to the coaxial longitudinal axis; pivoting of the pivotable head being to a first orientation with the inhalation aperture oriented radially from the longitudinal axis; sliding the pivotable head towards the housing second end along the longitudinal axis and extending of the heating element out from the housing first end; filling with phyto material extract a removable storage container bounded by at least two sidewalls forming a storage cavity; releasably coupling of the removable storage container with the housing so the storage cavity faces the housing, wherein extending of the heating element away from the housing first end parallel with the longitudinal axis comprises contacting phyto material extract contained within the storage cavity of the removable storage container. and wherein extending of the heating element away from the housing first end parallel with the longitudinal axis comprises contacting phyto material extract contained within the storage cavity of the removable storage container.

In some embodiments a vaporization device may include a predetermined vaporization temperature of between 500 degrees Fahrenheit and 800 degrees Fahrenheit.

In some embodiments a vaporization may include a pivotable head coupled with the slider assembly proximate the second end of the housing and for extending past the housing second end, the pivotable head for being pivotable about a transverse axis that is approximately perpendicular to the longitudinal axis; rotating of the pivotable head about the transverse axis and extending of the heating element away from the housing first end parallel with the longitudinal axis.

These and other aspects and features of various embodiments will be described in greater detail below.

### BRIEF DESCRIPTION OF THE DRAWINGS

For a better understanding of the described embodiments and to show more clearly how they may be carried into effect, reference will now be made, by way of example, to the accompanying drawings in which:

FIG. 1 is a top, front perspective view of an example vaporization device, in accordance with an embodiment;

FIG. 2 is a top, front perspective view of the example vaporization device of FIG. 1 with a housing removed;

FIG. 3 is a front perspective view of a heating chamber of the example vaporization device of FIG. 1 with a moveable member in a first position;

FIG. 4 is a front perspective view of the heating chamber of FIG. 3 with the moveable member in a second position;

FIG. 5 is a cut-away top plan view of the heating chamber of FIG. 3;

FIG. 6 is a cut-away side view of another example vaporization device, in accordance with an embodiment;

FIG. 7 is a cut-away side view of another example vaporization device, in accordance with an embodiment;

FIG. 8 is a cut-away side view of another example vaporization device, in accordance with an embodiment;

FIG. 9 is a perspective view of the example vaporization device of FIG. 8;

FIG. 10 is a perspective view of another example vaporization device, in accordance with an embodiment;

FIG. 11 is a side view of the example vaporization device of FIG. 10 showing the cylindrical conductive heating element in contact with phyto material provided on a surface;

FIG. 12 is a cut-away side view of the example vaporization device of FIG. 10;

FIG. 13 is a perspective view of another example vaporization device, in accordance with an embodiment;

FIG. 14 is a side view of the example vaporization device of FIG. 13 showing an example heating element in contact with phyto material, in accordance with an embodiment;

FIG. 15 is a side view of another example vaporization device showing another example heating element in contact with phyto material, in accordance with an embodiment;

FIG. 16 is a perspective view of another example vaporization device, in accordance with an embodiment;

FIG. 17 is a perspective view of another example vaporization device, in accordance with an embodiment;

FIG. 18 is side view of a vaporization device that includes a removable storage container as a removable heating chamber and in a first mode of operation;

FIG. 19 is top perspective view of a vaporization device shown in FIG. 18 that includes a removable storage container as a removable heating chamber and in a first mode of operation;

FIG. 20 is a side view of a vaporization device that includes a removable storage container being uncoupled from a housing and in a second mode of operation;

FIG. 21 is a top perspective view of a vaporization device that includes a removable storage container coupled from a housing and in a second mode of operation;

FIG. 22 is a bottom perspective view of a vaporization device that includes a removable storage container uncoupled from a housing and in a first mode of operation;

FIG. 23 is a side view of a vaporization device having a pivotable head;

FIG. 24 is a bottom perspective view of a vaporization device having a pivotable head with a heating element uncoupled from a slider assembly;

FIG. 25 is a top perspective view of a vaporization device having a pivotable head and showing a transverse axis;

FIG. 26 is a bottom perspective view of a vaporization device having a pivotable head with a heating element coupled to a slider assembly; and

FIG. 27 is a side view of a vaporization device having a pivotable head in a second orientation.

The drawings included herewith are for illustrating various examples of articles, methods, and apparatuses of the teaching of the present specification and are not intended to limit the scope of what is taught in any way.

### DESCRIPTION OF EXAMPLE EMBODIMENTS

Various apparatuses, methods and compositions are described below to provide an example of an embodiment of each claimed invention. No embodiment described below limits any claimed invention and any claimed invention may cover apparatuses and methods that differ from those described below. The claimed inventions are not limited to apparatuses, methods and compositions having all of the features of any one apparatus, method or composition described below or to features common to multiple or all of the apparatuses, methods or compositions described below. It is possible that an apparatus, method or composition described below is not an embodiment of any claimed invention. Any invention disclosed in an apparatus, method or composition described below that is not claimed in this document may be the subject matter of another protective instrument, for example, a continuing patent application, and the applicant(s), inventor(s) and/or owner(s) do not intend to abandon, disclaim, or dedicate to the public any such invention by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures, and components have not been described in detail so as not to obscure the example embodiments described herein. Also, the description is not to be considered as limiting the scope of the example embodiments described herein.

The terms “an embodiment,” “embodiment,” “embodiments,” “the embodiment,” “the embodiments,” “one or more embodiments,” “some embodiments,” and “one embodiment” mean “one or more (but not all) embodiments of the present invention(s),” unless expressly specified otherwise.

The terms “including,” “comprising,” and variations thereof mean “including but not limited to,” unless expressly specified otherwise. A listing of items does not imply that any or all of the items are mutually exclusive, unless expressly specified otherwise. The terms “a,” “an,” and “the” mean “one or more,” unless expressly specified otherwise.

Vaporization of phyto materials may emit vapors that may provide therapeutic benefits when inhaled by users. Devices used to heat phyto material to suitable vaporization temperatures are often referred to as vaporizers or vaporization devices.

Vaporization devices may include a heating chamber in which phyto material is heated and vaporized. The vaporization of the phyto material allows desired essential oils and other flavored extracts to be separated from the phyto material in vapor form and inhaled by a user of the device.

The heating time required by a vaporization device may impact the usability of the device. Devices with long heating times may result in delays between the time the device is activated and the vapor is emitted. This may negatively impact the user experience and may also result in additional drain on the device power source. For instance, the delay may result in a user leaving the device active for longer than necessary to ensure that the vaporization temperature has been reached, resulting in additional energy waste. Additionally, if phyto material is heated for a prolonged period of time, the potency of the desired essential oils and flavored extracts released as vapor may be reduced.

Controlling the heating of phyto materials placed in vaporization devices is also important for user experience and health. If phyto material is heated to its combustion temperature, the phyto material may combust and emit smoke rather than vapor. Careful control of the heat generated within the heating chamber may be required to ensure that the phyto material is vaporized rather than combusted.

Battery life may also impact usability of vaporization devices, particularly portable devices. Users may wish to use a portable vaporization device over the course of an extended period such as a day or multiple days. Individuals who use vaporization devices outdoors, or otherwise away from external power sources, may require a source of energy for an extended period of time. Vaporization devices with short battery life may not be suitable to such users. More generally, such vaporization devices may require more frequent charging or replacement of batteries, leading to more frequent periods when the device is not usable. This may further delay a user's ability to achieve the desired therapeutic effects.

Flexibility in recharging vaporization devices may be important, particular where the device is portable. Vaporization devices that use proprietary recharging connections may effectively prevent users from recharging the energy storage members while on the go, as there may not be suitable recharging stations accessible.

Embodiments described herein relate generally to vaporization devices. In general, the vaporization devices described herein may be used to vaporize phyto material derived from plant matter. In some cases, the vaporization devices described herein may also be used to vaporize materials extracted from phyto material, such a phyto material extracts and oils. Typically, phyto material extracts vaporize at higher temperatures than leaf phyto materials, for example from 500 t0 800 degrees Fahrenheit. Leaf phyto materials typically vaporize at temperatures of about 370 to 435 degrees Fahrenheit.

Various types of phyto material derived from plant matter may be vaporized for aromatherapy or medicinal treatment regimens. For instance, phyto material from cannabis plants, such as the buds and/or leaves, may be vaporized. A user may inhale the cannabis vapor generated from a vaporization device to achieve associated therapeutic effects.

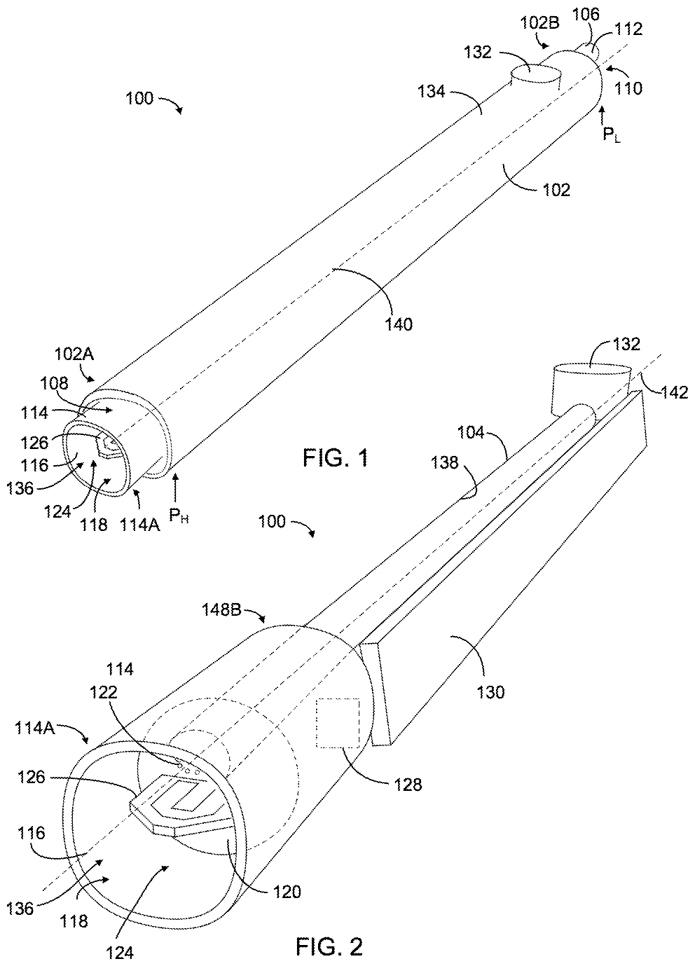

Referring generally to FIGS. 1-5, shown therein is an example of a vaporization device 100. FIG. 1 shows a perspective view of the vaporization device 100 including a housing 102. FIG. 2 illustrates the vaporization device 100 with the housing 102 removed to more clearly identify components positioned within the housing 102.

The housing 102 extends between a housing first end 102A and a housing second end 102B. The housing 102 may define a central axis 140 of the vaporization device 100.

In the example shown, the housing 102 defines a substantially cylindrically shaped vaporization device 100. This may provide a sleek profile for the vaporization device 100 that may improve portability and facilitate storage in a pocket. The circular cross-section of the vaporization device 100 may also provide a relatively strong exterior housing 102.

In some embodiments, the vaporization device 100 may include flattened or partially flattened sides. For instance, the housing 102 may be generally cylindrical in shape, with four or more partially flattened sides. Providing flattened, or partially flattened, sides may reduce the likelihood of the vaporization device 100 rolling off the edge of a table or other surface.

Alternatively, the vaporization device 100 may be rectangular or another suitable shape.

The housing 102 may enclose a fluid pathway 104 that extends between the housing first end 102A and the housing second end 102B. An inhalation aperture 106 is positioned at the housing second end 102B. The inhalation aperture 106 is fluidly coupled with the fluid pathway 104.

The first end 102A of the housing 102 may be an open housing end 108. Thus, the first end 102A may be exposed to an external environment.

In some cases, the second end 102B of the housing 102 may be a partially closed housing end 120. As shown, the second end 102B may be closed except for the inhalation aperture 106.

In the example shown, the inhalation aperture 106 extends from the closed housing end 120 to define a mouthpiece 122. Alternatively, the inhalation aperture 106 may be flush with the end 102B of the housing, e.g. an opening formed in the closed end 120 (see e.g. inhalation aperture 206 of FIG. 6).

Vaporization device 100 also includes a heating chamber 124 proximate to the housing first end 102A. The heating chamber 124 is in fluid communication with the fluid pathway 104. The heating chamber 124 may provide a phyto material receiving area in which phyto material may be positioned for vaporization.

As shown, the heating chamber 124 has a heating chamber first end 124A and a heating chamber second end 124B. A heating chamber wall 126 extends between the heating chamber first end 124A and the heating chamber second end 124B.

The first end 124A of the heating chamber 124 (also referred to as the outer end) may be an open chamber end 128. The open chamber end 128 may expose the heating chamber 124 and allow access to the phyto material receiving area. Having the first end 124A open also allows ambient air to be drawn into the heating chamber 124.

Optionally, the heating chamber 124 may include a closure member or lid. The closure member may be detachably attachable to the first end 124A of the heating chamber 124. This may allow the heating chamber 124 to be substantially closed when phyto material is positioned in the heating chamber 124 to be vaporized. The closure member may include one or more ambient air inlets to allow ambient air to be drawn into the heating chamber 124 when the closure member is positioned on the first end 124A.

The second end 124B of the heating chamber 124 (also referred to as the inner end) may be a substantially closed chamber end 120. The second end 124B of the heating chamber 124B may be closed (i.e. fluidly sealed) with the exception of one or more vents, or vapor inlets 122. Each vapor inlet 122 may extend through the closed chamber end 120. The vapor inlets 122 may fluidly couple the heating chamber 124 to the fluid pathway 104.

In some embodiments, the heating chamber 124 may be housed entirely within housing 102. For instance, the heating chamber first end 124A may not extend out from the housing first end 102A (see e.g., FIG. 6). The housing 102 may enclose the wall 126 of the heating chamber 124. In some embodiments, the inner surface of the housing 102 may provide the wall 126 of the heating chamber 124.

Alternatively, the housing 102 may provide an external insulation layer exterior to the wall 126. This may reduce the likelihood of a user burning themselves as a result of the heating chamber 124 being hot when vaporizing phyto material.

Alternatively, the heating chamber 124 may protrude at least partially from the first end 102A of the housing 102 as shown. This may promote only the heating chamber 124, and components therein, coming into direct contact with the phyto material.

The heating chamber 124 may also define a phyto material receiving area. The phyto material receiving area of vaporization device 100 may be defined as a volume bounded by the chamber wall 126, the heating chamber first end 124A and the heating chamber second end 124B. As shown, the phyto material receiving area may be an internal heating chamber cavity into which phyto material may be positioned. Phyto material to be vaporized may be loaded into the internal cavity 124 through the open chamber end 128.

Vaporization device 100 also includes a heating element 126. The heating element 126 is operable to heat phyto material to a predetermined vaporization temperature. The predetermined vaporization temperature may vary depending on the type of phyto material being vaporized. For instance, the vaporization temperatures for various cannabis phyto materials and extracts that may typically be vaporized may range between about 300 degrees Fahrenheit to about 450 degrees Fahrenheit.

In some embodiments, the heating element 126 may be heatable to temperatures that range from about 330 degrees Fahrenheit to about 900 degrees Fahrenheit. This may also allow the heating element 126 to perform a cleaning function in which excess or spent phyto material may be incinerated.

The heating element 126 may be configured to generate heat at the predetermined vaporization temperature. As shown in FIG. 2, the conductive heating element 126 may extend into the heating chamber 124. The heating element 126 may extend from the second chamber end 124B into the internal cavity 124 of the heating chamber 124. The heating element 126 may operate to vaporize phyto material positioned within the heating chamber 124.

The heating element 126 may be positioned centrally within the heating chamber 124. As shown in FIG. 2, the heating element 124 may extend along a plane that includes the central axis 140 of the vaporization device 100. The conductive heating element 126 may be configured to outwardly radiate heat within the internal cavity 124 toward the chamber wall 126. This may allow the heating element 126 to more evenly heat the heating chamber 124.

In the illustrated example, phyto material may be loaded into the phyto material receiving chamber 124 between the heating element 126 and the chamber wall 126. Outwardly radiating heat at the predetermined vaporization temperature, generated from the heating element 126, may at least partially vaporize the phyto material. Phyto material vapor may then be emitted and drawn into the fluid pathway 104.

The heating element 126 may be a conductive heating element that is heatable using electrical current. The heating element 126 may be electrically connected to a power source, such as an onboard energy storage member. Current from the power source may be discharged through the heating element 126 which may dissipate the energy into the heating chamber 124 as heat. The heating element 126 may radiate heat into the heating chamber 124, and into any phyto material in contact therewith (or in proximity thereto).

Vaporization device 100 may also include an energy storage member 128. The energy storage member 128 may be one or more batteries or a battery pack. The energy storage member 128 may be electrically coupled to the conductive heating element 126.

Vaporization device 100 may also include a control circuit 130 electrically coupled to the energy storage member 128. Control circuit 130 may be configured to control the flow of electrical current from the energy storage member 128 to the conductive heating element 126. The control circuit 130 may be implemented using various control components, such as a microprocessor, FPGA and/or application specific circuitry.

As shown in FIGS. 1 and 2, the energy storage member 128 and control circuit 130 may be disposed within the housing 102. The housing 102 may fully enclose the energy storage member 128 and the control circuit 130. The housing 102 may prevent dirt or debris from clogging the energy storage member 128 and the control circuit 130, which may be particularly useful when the vaporization device 100 is used outdoors. The housing 102 may also provide some protection from damage due to impacts.

Alternatively, the battery 128 and/or control circuit 130 may be at least partially exposed by the housing 102. This may provide easier access to the battery 128 or control circuit 130 for repair or replacement.

In the illustrated example, the vaporization device 100 includes a switch 132. The switch 132 may be user-activated to control the flow of current from the battery 128 to the conductive heating element 126. For instance, the switch 132 may be implemented using a mechanical switch, a hall-effect switch, or even a touchscreen display. In some cases, the switch 132 may be activated in response to detecting air flow through the vaporization device 100, e.g. as a result of a user inhaling from the inhalation aperture 106.

As shown in FIG. 1, the user activated-switch 132 may protrude from the housing 102. Alternatively, however, the switch 132 may be flush with the surface of housing 102, or even recessed therein.

As shown in FIG. 1, the switch 132 may be positioned proximate the housing second end 1026. This may allow a user to hold the vaporization device 100 near the inhalation aperture 106 and operate the switch 132 using the same hand.

Alternatively, user-activated switch 132 may also be positioned elsewhere alone the length of the vaporization device (see e.g., user-activated switch 232 of FIG. 6).

Optionally, the vaporization device 100 may include additional control and configuration inputs and/or outputs. For instance, the vaporization device 100 may include a temperature output indicating the predetermined vaporization temperature. In some embodiments, the predetermined vaporization temperature may be adjustable using a temperature setting input, such as a touchscreen, switch or dial.

The switch 132 may be moveable between an active position and an inactive position. When the switch is in the active position, the flow of current from the battery 128 to the conductive heating element 126 may be enabled. When the switch is in the inactive position, current may be prevented from flowing from the battery 128 to the conductive heating element 126.

The switch 132 may be connected to control circuit 130. The control circuit 130 may control the flow of current from the battery 128 to the heating element 126 to ensure that the heating element 126 is heated to the predetermined vaporization temperature.

In some embodiments, the switch 132 may activate the control circuit 130 when moved to the active position. When the control circuit 130 is active, the control circuit 130 may permit current to flow to the heating element 126.

In some embodiments, activating the switch 132 may cause the control circuit 130 to direct current from the battery 128 to the heating element 126. Alternatively, the control circuit 130 may selectively provide currently to the heating element 126, e.g. in response to sensor readings from an airflow or puff sensor as described herein below.

In the illustrated example, when current is provided to the heating element 126, the conductive heating element 126 may heat phyto material received within the heating chamber 124. When the phyto material is heated to the predetermined vaporization temperature, a phyto material vapor is emitted.

In operation, a user may inhale from the inhalation aperture 106. In response to a user inhaling from the inhalation aperture 106, a pressure gradient may be created across the fluid pathway 104. As shown is FIG. 1, a reduced pressure region P_{L} may result at the housing second end 102B. This reduced pressure region P_{L} is a region of lower pressure as compared to the pressure P_{H} at the housing first end 102A.

A user inhaling from inhalation aperture 106 may draw ambient air 136 from the external environment into the heating chamber 124 through open chamber end 128. As it flows through the heating chamber 124, the ambient air 136 may mix with vapor emitted from the phyto material being vaporized. The mixed vapor and air is drawn by the pressure gradient through the fluid pathway 104 to the inhalation aperture 106. The mixed vapor and air then exits the vaporization device 100 and may be inhaled by the user through inhalation aperture 102.

Optionally, the vaporization device 100 may include a thermally conductive section coupled to the fluid pathway 104. The thermally conductive section may be proximate the inhalation aperture 104, near the housing second end 102B. The thermally conductive section may provide heat transfer between ambient air external to the vaporization device 100 and the mixed vapor and air flowing through the fluid pathway 104. This may cool the mixed vapor and air as it approaches the inhalation aperture 106, so that the mixture inhaled by a user is not so hot that it scalds or injures them.

Optionally, an inner portion 138 of the fluid pathway 120 may include a thermally conductive liner. This conductive liner may also serve to reduce the temperature of the vapor and air flowing through fluid pathway 104.

The thermally conductive portion may include a section of material that provides greater thermal conductivity from the rest of housing 102. For instance, housing 102 may be formed generally using glass materials, while the thermally conductive portion may include metallic materials.

In some embodiments, the vaporization device 100 may include one or more sensors operable to monitor the flow of air through the fluid pathway 104. For example, an airflow sensor (not shown) may be positioned within the fluid pathway 104. The airflow sensor may operate to detect air flow through the fluid pathway 104. In some embodiments, the airflow sensor may also detect an airflow rate (i.e. the volume of air/vapor being drawn through the fluid pathway per unit time) through the fluid pathway 104.

The airflow sensor may be electronically coupled to the control circuit 130. The airflow sensor may transmit airflow signals to the control circuit 130. In some embodiments, the airflow signals from the airflow sensor may be used to trigger the supply of current to the heating element 124.

The control circuit 130 may be configured to monitor the airflow through the fluid pathway 104 in response to the airflow signals from the airflow sensor. For example, the control circuit 130 may detect airflow through the heating chamber 124 using the signals from the airflow sensor. In some cases, the control circuit 130 may determine the volume of ambient air 136 being drawn through the heating chamber 124.

In some embodiments, the control circuit 130 may initiate heating of the heating element 126 in response to signals from the airflow sensor. For example, the control circuit 130 may provide current to the heating element immediately in response to detecting airflow through the fluid pathway 104. This may ensure that the phyto material may be vaporized when a user is inhaling from the vaporization device 100, without over-heating the phyto material or having a prolonged heating period. In some embodiments, the control circuit 130 may also cease the provision of current to the heating element 124 when airflow is no longer detected or if the airflow rate drops below a threshold value.

In some embodiments, the heating element 126 of vaporization device 100 may be heated in response to signals from the airflow detector. For instance, the switch 132 may operate as an on/off switch activating the control circuit 130, and the control circuit 130 may provide current to heating element 126 in response to the detection of a user inhaling from the vaporization device 100.

A time required for the heating element 126 to be heated to a predetermined vaporization temperature may vary depending on the specific configuration of the heating element 126. Accordingly, it may be preferable for the heating element 126 to be activated in response to airflow signals only in embodiments in which the heating element 126 may be heated sufficiently rapidly to vaporize phyto material while the user continues to draw in air from inhalation aperture 106.

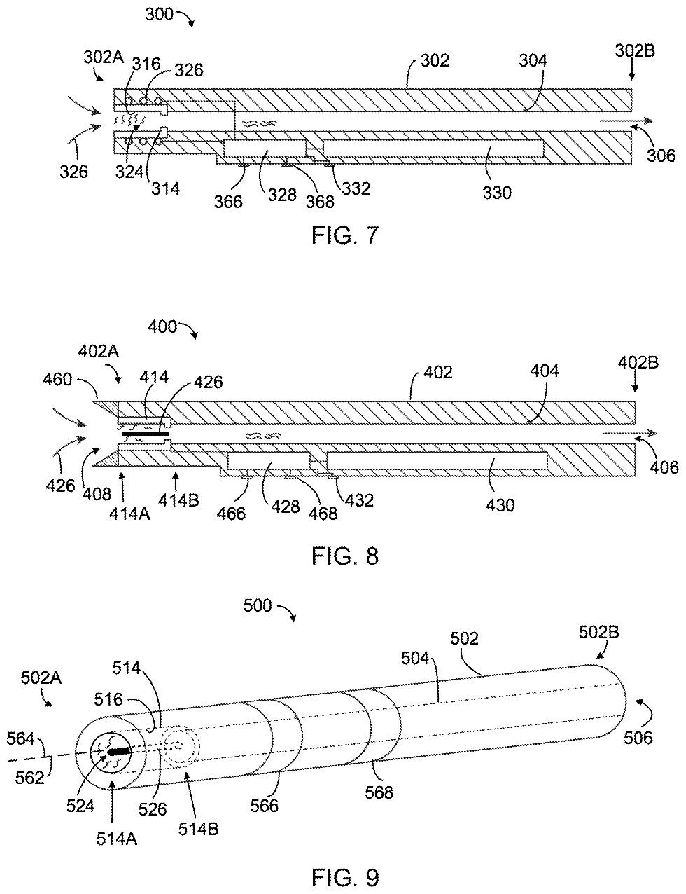

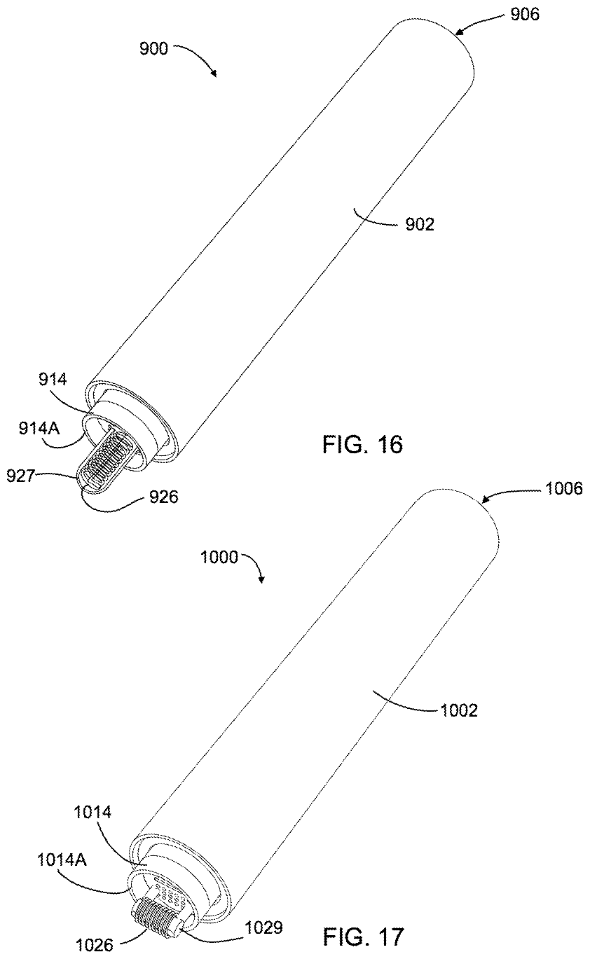

For example, the inventor has found that ceramic plate heating elements (such as those shown in FIGS. 1-5, 13 and 14) with a heating element resistance of 0.5 Ohm and a voltage of 7V applied across the heating element may be heated to about 500 F to 700 F in about 8 to 10 seconds. The inventor has also found that a rod (spike) heating element (such as those shown in FIGS. 7, 8, and 9) with 0.2 Ohm heating element resistance and a voltage of 7V applied across the heating element may be heated to about 500 F to 700 F in about 3 to 5 seconds. Using a coil heating element (such as those shown in FIGS. 16-17) with a resistance of about 0.1-0.2 Ohms and applying a voltage of 3.7V across the heating element, the inventor has found that the heating element may be heated to about 500 F to 700 F in about 1.5 to 5 seconds. Accordingly, it may be preferable to use coil-based heating elements or rod heating elements when the heating elements are heated in response to a user inhaling. In some embodiments a thinner wire (higher gauge) and a higher resistance may be used and combinations of wire thickness and resistance for providing optimal heating times, as would be obvious to one of skill in the art.

In some embodiments, the control circuit 130 may also use the airflow sensor signals to monitor the state of the phyto material positioned in the heating chamber 124. For example, the heating chamber 124 may be sized to accommodate a defined volume of phyto material. By monitoring a volume of ambient air drawn through the heating chamber 124 and the predetermined vaporization temperature of the heating element 126, the control circuit 130 may determine that phyto material positioned within the heating chamber 124 has been substantially vaporized. The control circuit 130 may then provide an indication to the user (e.g. a visible or audible signal) that the phyto material in the heating chamber 124 may need to be replaced.

In some embodiments, the control circuit 130 may be configured to monitor a dose of phyto material consumed by a user of the vaporization device 100. The control circuit 130 may estimate a dose consumed by a user based on the size of the phyto material receiving area and the airflow through the heating chamber 124 when the phyto material is heated to a vaporization temperature. In some cases, a predefined quantity of phyto material may be inserted into the internal cavity 124 of the heating chamber 124, and the control circuit 130 may monitor the airflow through the heating chamber 124 to estimate the dose consumed by a user.

In some embodiments, the vaporization device 100 may include a temperature sensor (not shown). The temperature sensor may be positioned proximate to, or within, the heating chamber 124 to measure a temperature of the internal cavity 124.

The temperature sensor may be electronically coupled to control circuit 130. The temperature sensor may be configured to transmit temperature readings to the control circuit 130. The control circuit 130 may adjust the flow of electric current from the battery 128 to the conductive heating element 126 based on the received temperature readings to retain the heating chamber 124 at the predetermined vaporization temperature.

In some embodiments, the control circuit 130 may provide an indication to the user (e.g., a visible or audible output) that the predetermined vaporization temperature has been reached. This may alert the user that vapor is being emitted and may be inhaled. In some embodiments a TCR (temperature coefficient of resistance) may be utilized in order to approximate (using a lookup table or a formula) a temperature of the heating element.

In the example shown in FIG. 1, the housing 102 extends axially along a central axis 140. The fluid pathway 104 extends axially along a fluid pathway central axis 142 that extends generally between the housing first end 102A and the housing second end 102B. In the illustrated example, the central axis 142 of the fluid pathway 104 is offset from the central axis 140 of the housing 102. Offsetting the fluid pathway 104 within the housing 102 may allow the battery 128 and control circuit 130 to occupy more than 50% of the space within the housing 102. This may provide a more compact vaporization device 100, as the fluid pathway 104 may occupy less than 20% of the vertical space within the housing 102 for the majority of the longitudinal length of the housing 102. Furthermore, this provides for a relatively straight fluid pathway 104 and this facilitates ease of cleaning of the fluid pathway 104.

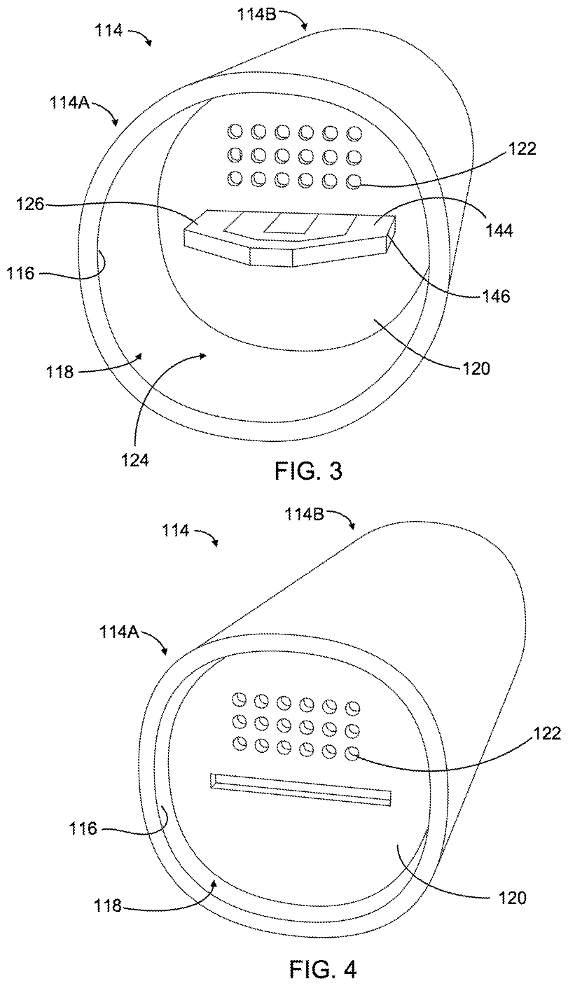

Alternatively, the housing central axis 140 and the fluid pathway central axis 142 may be co-axial (see e.g. FIG. 9). As shown in FIG. 3, the heating element 126 may extend into the internal cavity of the heating chamber 124. The heating element 126 may be positioned centrally within the heating chamber 124. This may allow the heating element 126 to contact phyto material positioned through the heating chamber 124. By positioning the heating element 126 in direct contact with phyto material, the phyto material may be heated more efficiently.

In some embodiments, the heating element 126 may also be thermally coupled to the chamber wall 126. For example, the second end 124B of the housing 124 may be formed of a thermally conductive material. The second end 124B may transfer heat from the heating element 126 to the chamber wall 126. The chamber wall 126 may, in turn, heat phyto material positioned within the heating chamber 124.

As shown in FIG. 3, the conductive heating element 126 may be a blade-shaped heating element. In the illustrated example, the blade-shaped heating element may be aligned centrally within the internal cavity 124 of the heating chamber 124. Heat generated by the blade-shaped heating element may radiate outwardly toward the chamber wall 126.

The heating element 126 may be manufactured of various materials. For instance, the heating element may be manufactured of ceramic materials, such as alumina ceramic. In some cases, the heating element 126 may be manufactured using high-temperature co-fired ceramics. For example, the heating element 126 may comprise a combination of high melting point metal heating materials such as tungsten, molybdenum, and/or molybdenum-manganese along with alumina ceramic substrates. The heating element 126 may be formed using a metal heating resistance slurry that is printed onto a ceramic green body in the desired configuration. This combination may be fired at a temperature of about 1500˜1600 degrees Celsius along with a sintering additive, to form an alumina ceramic heating element. In some embodiments the heating element may be formed from a stamped metal and affixed to a substrate.

In the illustrated example, the heating chamber 124 may be manufactured using ceramic materials. In some embodiments, chamber wall 126 is a ceramic material while the closed chamber end 120 may be metallic. Manufacturing the closed chamber end 120 using metallic materials may facilitate formation of the one or vents 122. In some embodiments, the heating chamber 124 may be formed with a ceramic exterior layer and the housing element 126 formed there within.

The housing 102 may be manufactured using various material, such as metallic materials. For instance, the housing 102 may be manufactured of gold-plated copper, anodized aluminum or a TiN plated metal. In some cases, the housing 102 may be thermally conductive. This may allow the housing 102 to provide heat transfer between the fluid pathway 104 and the external environment.

In the example illustrated in FIG. 3, the blade-shaped heating element is a substantially planar heating element. The blade-shaped heating element 126 includes two parallel flat long sides 144 and two parallel flat narrow sides 146. In this configuration, a large portion of the heat radiates from the two parallel flat long sides 144 and outwardly toward the chamber wall 140. By providing the heating element 126 with an extended surface area along the long sides 144, the surface area in contact with the phyto material may be increased while still providing a large phyto material receiving space.

Alternatively, the heating element may be a circular or other shaped heating element extending into the heating chamber 124. For instance, the heating element may be shaped as a cylindrical rod or spike (see e.g. FIGS. 6, 8, and 9).

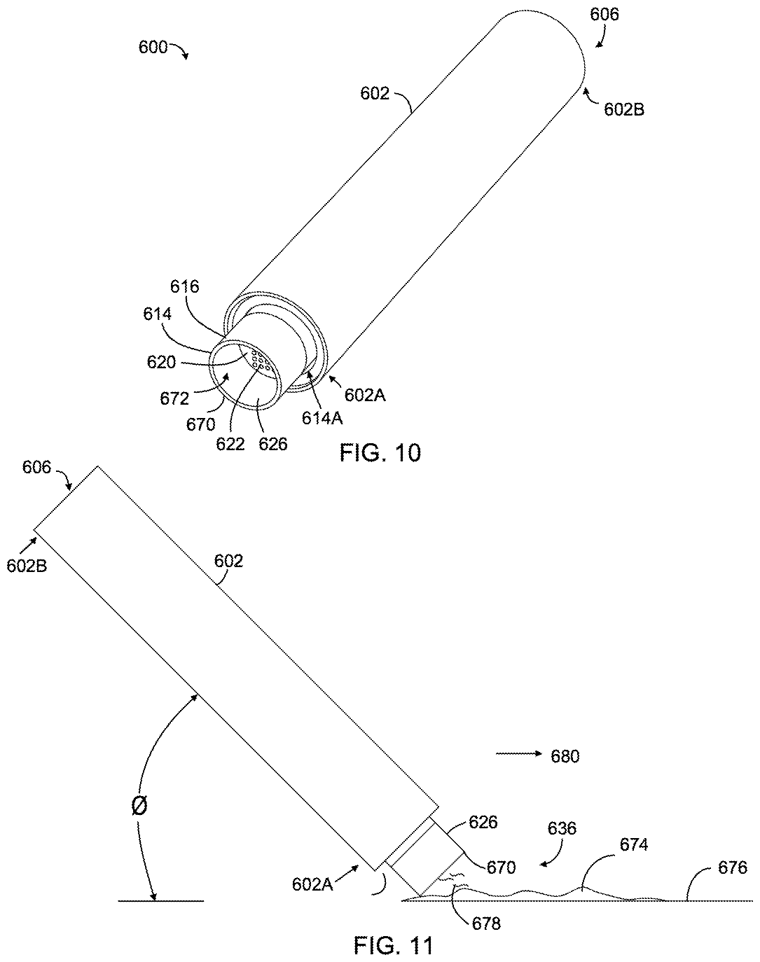

Alternatively, the heating element may be positioned to contact the chamber wall 126 of the heating chamber 124 (see e.g. FIG. 7) or integrated with the chamber wall 126 (see e.g. FIG. 10). For instance, a cylindrical heating element may be formed on the inner surface of the chamber wall 126, or embedded into the chamber wall 126. Alternatively, the heating element may be positioned to contact an outer surface of the chamber wall 126, and the chamber wall 126 may transfer the heat from the heating element to the phyto material positioned within the heating chamber 124.

In some embodiments, the heating chamber 124 may include a sliding member. In the example shown by FIGS. 3-4, the sliding member may be defined by the closed end 120 of the heating chamber 124. The sliding member may be moveable between a first position (also referred to as a load position), an example of which is shown in FIG. 3, and a second position (also referred to as an eject position), an example of which is shown in FIG. 4.

In the first position, the sliding member 120 may be positioned proximate to the heating chamber second end 124B. This may provide a user with access to the phyto material receiving area, e.g. to allow phyto material to be loaded and/or to facilitate cleaning.

In the second position, the sliding member 120 may be positioned proximate to the heating chamber first end 124A. The sliding member 120 may transition from the first position to the second position to eject phyto material from the phyto material receiving area. The second position may also facilitate cleaning the closed end 120 of the heating chamber 124, e.g. by providing easy access to vapor inlets 122.

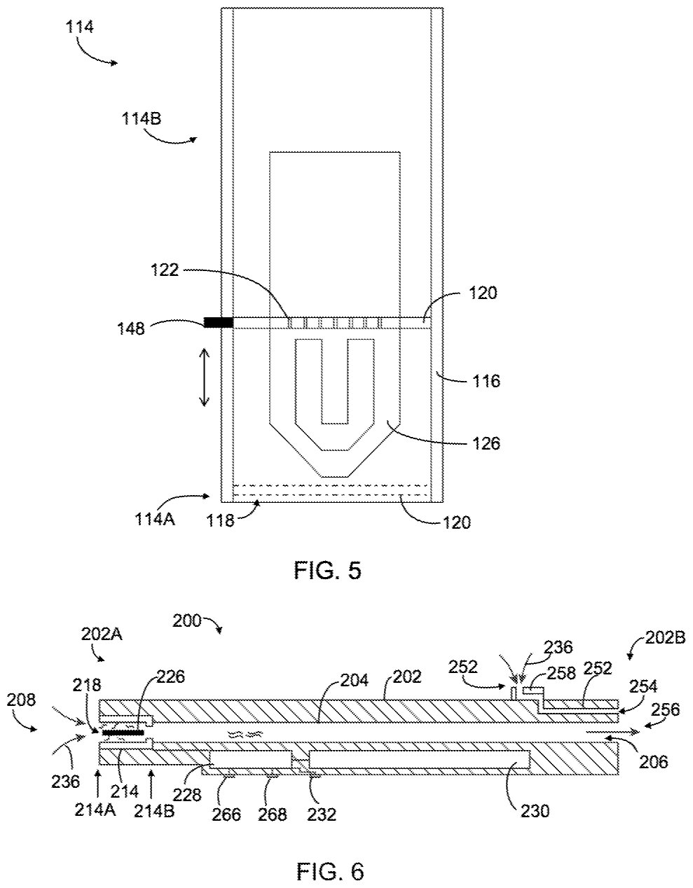

As shown in the example of FIG. 5, the sliding member 120 may be manually adjustable between the first position and the second position. An actuator 148 may be provided on the vaporization device 100 to allow a user to move the sliding member between the first and second positions. For instance, the actuator 148 may be a switch or paddle.

In some embodiments, the sliding member 120 may be biased to the first position. For instance, the actuator 128 may include a spring-loaded trigger. A user may adjust the sliding member 120 to the second position when ejecting phyto material. The sliding member 120 may then automatically retract to the first position in the absence of user intervention.

In some embodiments, the sliding member 120 may provide a cover for the heating element 126. As shown in FIGS. 3 and 4, the sliding member 120 may also include a void or slot corresponding to the heating element 126. Thus, the heating element 126 may remain stationary when the sliding member 120 is moved between the first and second positions. This may facilitate construction, as the sliding member 120 may not include any active components.

The sliding member 120 may also protect the heating element 126 when the vaporization device 100 is not in use. For instance, the sliding member 120 may be adjustable to the closed/eject position when the device is not being used so that the heating element 126 is recessed behind sliding member 120.

As described above, the closed chamber end 120 includes one of more vents or vapor inlets 122. In the illustrated example, the heating chamber 124 includes eighteen vents 122. However, various other numbers and arrangements of vapor inlets may be provided in the heating chamber 124. In some embodiments pores of various sizes may be provided for facilitating of vapor to propagate through the fluid pathway 104 and for substantially inhibiting phyto material from being drawn into the fluid pathway 104. The vents 122 may be sized to inhibit phyto material from being drawn into the fluid pathway 104. In some cases, a screen or filter may be provided to prevent phyto material from entering the fluid pathway 104.

In some embodiments, the vapor inlets 122 may be provided only on a first side of the heating element 126 as shown in FIGS. 3 and 4. This may allow the fluid pathway 104 to be offset from the central axis 140 of the vaporization device 100 throughout the length of vaporization device 100.

Alternatively, vapor inlets 122 may be distributed around the heating element 122. This may facilitate drawing vapor and ambient air into the fluid pathway 104.

In some embodiments, the vaporization device 100 may also include a cleaning setting. The cleaning setting may facilitate discarding phyto material from the heating chamber 124. When the cleaning setting is activated (e.g. by a user activating a cleaning input setting), the control circuit 130 may operate to heat the heating element 126 to a combustion temperature of the phyto material (e.g. a temperature of 600 to 900 degrees Fahrenheit or greater, or 750 degrees Fahrenheit or so). The heating element may then incinerate phyto material debris positioned in the heating chamber that may otherwise be stuck on the inner surfaces of the heating chamber 124 or heating element 126. The incinerated phyto material may then be removed from the vaporization device 100, e.g. manually or using a sliding member 120, or through a brushing process.

FIG. 6 illustrates another example vaporization device 200 in accordance with an embodiment. Elements having similar structure and/or performing similar function as those in the example vaporization device 100 in FIGS. 1 to 2 are numbered similarly, with the reference numerals incremented by 100.

As shown in the example of FIG. 6, a housing 202 encloses a primary fluid pathway 204. The primary fluid pathway 204 extends axially between a housing first end 202A and a housing second end 202B. A primary inhalation aperture 206 is formed through the housing 202 at the housing second end 202B. The primary inhalation aperture 206 is in fluid communication with the primary fluid pathway 204.

Vaporization device 200 also includes a secondary air input port 250 coupled to a secondary fluid pathway 252. The secondary fluid pathway 252 extends between the secondary air input port 250 and the housing second end 202B.

In the example shown, vaporization device 200 includes a secondary inhalation aperture 254 positioned proximate the housing second end 202B. The secondary inhalation aperture 254 may be positioned adjacent to the inhalation aperture 206. A user inhaling from the device 200 may then draw air through both the primary inhalation aperture 206 and secondary inhalation aperture 254.

The secondary inhalation aperture 254 and primary inhalation aperture 206 may be joined in a common mouthpiece at the housing second end 2028. This may ensure that a user draws air through both the primary inhalation aperture 206 and secondary inhalation aperture 254.

In some embodiments, the secondary fluid pathway 252 may join the primary fluid pathway 204 upstream from the inhalation aperture 206. This may allow the device 200 to include only a single inhalation aperture.

As shown in FIG. 6, the primary fluid pathway 204 may be positioned centrally within the housing 202. This may facilitate providing a secondary fluid pathway 252 within housing 202.

Alternatively, the primary fluid pathway 204 may be offset from the central axis of housing 202 (as described above). In some such embodiments, the secondary fluid pathway 252 may join the primary fluid pathway 204 proximate the inhalation aperture 206 so that the secondary fluid pathway 252 may also be entirely contained within housing 202.

In some cases, offsetting the primary fluid pathway 204 may allow the secondary fluid pathway 252 to be formed on the opposite side of the vaporization device 200.

The vaporization device 200 may include a puff sensor 258 positioned in the secondary fluid pathway 252. The puff sensor 258 may operate to detect airflow through the secondary fluid pathway 252. For instance, the puff sensor 258 may be a pressure-based sensor that detects a pressure gradient caused by a user inhaling from the vaporization device 200. A secondary pressure gradient may be created across the secondary fluid pathway 252.

The secondary pressure gradient may draw a volume of ambient air 236′ from the external environment into the ambient air input port 250 that is separate from the volume of ambient air 236 drawn into the heating chamber 214. In response to detecting the ambient air 236, the puff sensor 258 may transmit a puff signal to the control circuit 230. The control circuit 230 may control the flow of the electric current from the battery 228 to the conductive heating element 226 in response to signals from the puff sensor 258.

For example, the puff sensor 258 may detect a user inhalation. The control circuit 230 may then activate the heating element 226 in response to detecting the inhalation in order to generate phyto material vapor.

Providing a separate airflow path 252 for the puff sensor 258 may ensure that the inhalation is detected regardless of the status of the phyto material in the heating chamber 214 (i.e. even if the phyto material prevents or restricts airflow therethrough). Additionally, providing the puff sensor proximate the inhalation aperture 206 as shown may further reduce the time required to identify a user inhalation.

In some embodiments, the volume of ambient air 236′ passing through the secondary fluid pathway 252 may trigger the pressure-based puff sensor 258 to monitor the flow rate of the second volume of ambient air 236′. This may be used to monitor the duration and strength of inhalation from a user.

As shown in FIG. 6, the vaporization device 200 may also include a first battery contact 266 and a second battery contact 268. The first battery contact 266 and the second battery contact 268 may be electrically coupled to the control circuit 230.

The contacts 266/268 may be engageable with corresponding contacts of a battery recharging hub. The recharging hub may be connected to an external power source or may contain a battery of its own, or both. The recharging hub may provide electrical energy from the recharging contacts to the battery 230 via battery contacts 266, 268.

As shown, the battery contacts 266, 268 may protrude from the housing 202. Alternatively, the battery contacts 266, 268 may be providing flush with the housing 202.

Alternatively, charging ports may be provided on the vaporization device 200. For instance, the vaporization device 200 may incorporate a micro-USB or USB-C charging port to allow the energy storage member 228 to be recharged.

Optionally, a recharging unit may also be provided that corresponds to the vaporization device 100/200. The recharging may include a secondary housing within which the vaporization device may be positioned for charging. For instance, an example of a recharging unit that may be used in embodiments herein is described in further detail in U.S. patent application Ser. No. 14/829,660 the entirety of which is incorporated herein by reference.

FIG. 7 illustrates another example vaporization device 300 in accordance with an embodiment. Elements having similar structure and/or performing similar function as those in the example vaporization device 100 in FIGS. 1 to 2 are numbered similarly, with the reference numerals incremented by 200.

Vaporization device 300 is an example of a vaporization device employing a heating element 336 coupled to the chamber wall 316. As shown in FIG. 7, a conductive heating element 336 is positioned around the chamber wall 316 of the heating chamber 314. In some cases, the heating element 336 may be embedded within the chamber wall 316. The conductive heating element 336 is configured to heat the internal cavity 324 of the heating chamber 314 by heating the chamber wall 316. The chamber wall 316 may then transfer the heat to phyto material positioned within the heating chamber 314. This may provide the heating chamber 314 with a simplified structure that may be more easily cleaned.

FIG. 8 illustrates another example vaporization device 400 in accordance with an embodiment. Elements having similar structure and/or performing similar function as those in the example vaporization device 100 in FIGS. 1 to 2 are numbered similarly, with the reference numerals incremented by 300.

The vaporization device 400 is an example of a vaporization device employing a cylindrical heating chamber 414. The cylindrical heating chamber 414 may be positioned within the open housing end 408 such that the heating chamber first end 414A is aligned with the housing first end 402A and the heating second chamber end 414B is displaced from the housing first end 402A toward the housing second end 402B.

The vaporization device 400 may also be used as a phyto material preparation device. As shown in FIG. 8, the heating first chamber end 414A has a sharpened or pointed outer edge 460. The pointed edge 460 may be usable to cut or separate phyto material that is to be loaded into the heating chamber 414.

To prepare phyto material for loading, a user may hold the vaporization device 400 like a wand and position the pointed edge 460 to contact the phyto material. The edge 460 may be sharpened so that when pressed against the phyto material, the phyto material may tend to separate.

In some embodiments, the pointed edge 460 may be removably coupled to the heating chamber first end 414A. For example, the pointed edge 460 may be removed for sharpening or when it is otherwise not needed (e.g. when phyto material is loaded in the heating chamber 414).

FIG. 9 illustrates another example vaporization device 500 in accordance with an embodiment. Elements having similar structure and/or performing similar function as those in the example vaporization device 100 in FIGS. 1 to 2 are numbered similarly, with the reference numerals incremented by 400.

The vaporization device 500 includes a cylindrical heating chamber 514. A heating element 526 extends into the heating chamber 514. In the example of FIG. 9, the heating element 526 is a cylindrical rod-shaped heating element 526.

As shown in FIG. 9, the rod-shaped heating element 526 is aligned centrally within the internal cavity 524 of the heating chamber 514. That is, a heating chamber central axis 562 and a heating element central axis 564 are co-axial. As a result, the rod-shaped heating element is equally spaced from the chamber ball 516. In the illustrated example, the rod-shaped heating element 526 may be configured to radiate heat outwardly toward the chamber wall 516.

FIGS. 10-12 illustrate another example vaporization device 600 in accordance with an embodiment. Elements having similar structure and/or performing similar function as those in the example vaporization device 100 in FIGS. 1 to 2 are numbered similarly, with the reference numerals incremented by 500. In some embodiments the use of the vaporization device 600 is preferably with phyto material extracts, which are heated to higher temperatures than loose leaf phyto materials.

Vaporization device 600 includes a heating chamber 614. The heating chamber 614 defines a cylindrical heating element 626. The heating element 626 may include electrical coils 615 positioned at a second end 614A of the heating chamber 614. The electrical coils 615 may be positioned to contact the chamber wall 616. Alternatively, the coils may be embedded within the chamber wall 616. The coils may operate to heat the chamber wall 616 to provide the heating element 626.

In the illustrated example, an outer edge 670 of the cylindrical heating element 626 protrudes a distance Dₓ from the housing first end 602A. As shown in FIG. 10, an interior 672 of the heating chamber 614 is fluid communication with a fluid pathway that extends from the first end 602A of the housing 602 to an inhalation aperture 606 positioned at the second end 602B of the housing 602. The closed end 620 of the heating chamber 614 may include one or more vents or vapor inlets 622 coupling the heating chamber 614 to the fluid pathway.

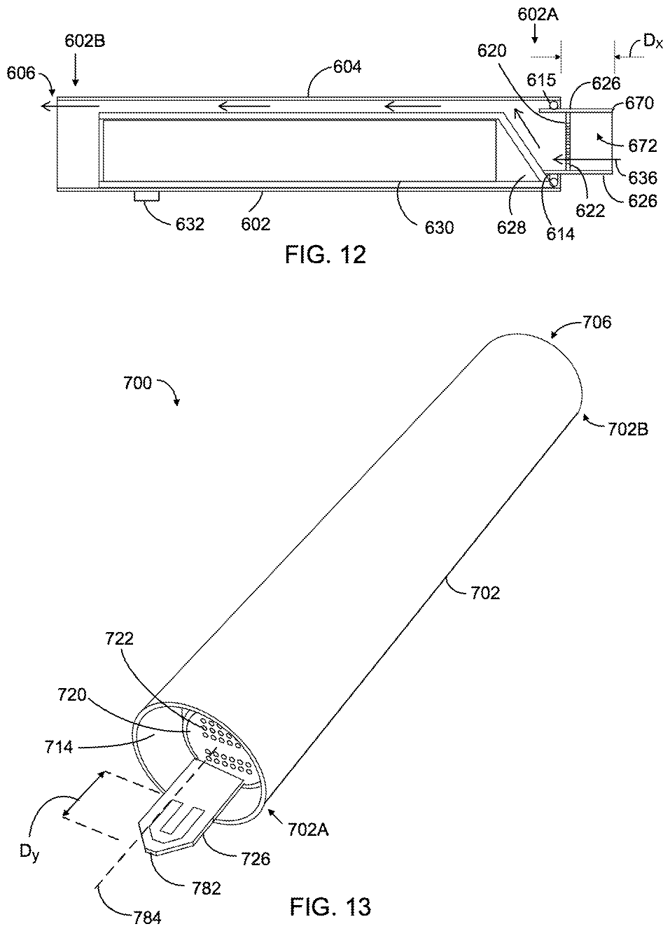

In use, the outer edge 670 of heating element 626 may be heated to a predetermined vaporization temperature. The outer edge 670 may be positioned in contact with a phyto material or phyto material extract 674 to vaporize the phyto material. For instance, phyto material may be positioned on a surface 676 (see FIG. 12) and the vaporization device 600 may be moved into contact with the phyto material to instigate vaporization. Thus, the phyto material extract need not be loaded into the vaporization device 600.

Vaporization device 600 may be used to vaporize phyto material extract that is not positioned within the heating chamber 614. Rather, the outer edge 670 of the heating element 626 may be placed in direct contact or proximate contact with external phyto material extract to induce vaporization. The heating chamber 614 may then act as an inhalation chamber that captures the vapor emitted and directs it to the fluid pathway and through to the inhalation aperture 606.

Vaporizing phyto material extract while external to vaporization device 600 may prevent the extract from becoming clogged in the heating chamber 614 and/or vents 622. This may reduce the amount of cleaning required for vaporization device 600 and may prolong the usable life of the vaporization device 600.

As shown in the example of FIG. 12, the vaporization device 600 may be used in a manner akin to a straw. A user may position the inhalation aperture 606 in their mouth and the outer edge 670 of the cylindrical heating element 626 on the phyto material 674 provided on the surface 676. The vaporization device 600 may be orientated at an angle θ to the surface in order to permit ambient air 636 from the external environment to be drawn into the cylindrical heating element 626. Preferably, the vaporization device 600 may be held at an angle between 40 and 90 degrees measured from the surface 676 to encourage the vapor to rise into the vaporization device 600.

The battery 628 may provide electric current to the coils 615 to heat the cylindrical heating element 626. For instance, the battery 628 may provide current in response to activation of the user-activated switch 632 and/or detection of airflow using an airflow or puff sensor as described herein above. In cases when the puff sensor is used then the heating element 626 may be manufactured from a low thermal inertia to facilitate quick heating of about a second or so. In some embodiments it may be preferable to have the user-activated switch 632 in order to facilitate heating of the heating element 626 to a predefined vaporization temperature of about 632 degrees Fahrenheit and then to provide a notification to the user when that temperature has been approximately achieved.

The cylindrical heating element 626 may heat the phyto material extract 674 to the predefined vaporization temperature, such as 500 degrees Fahrenheit, or to around 600 degrees Fahrenheit. In the illustrated example, the outer edge 670 of the cylindrical heating element 626 contacts and heats the phyto material extract 674 provided on the surface 676. When the temperature of the phyto material extract exceeds its vaporization temperature, the phyto material extract 674 emits a vapor 678. A user may then inhale through the vaporization device 600 as described herein above. A diameter of the cylindrical heating element 626 may be envisaged to be about 5 mm or 7 mm or in some cases to be about 6 mm.

During inhalation, the user may slide the vaporization device 600 along the surface 676 (e.g. in a direction 680) that has on its surface disposed the phyto material extract 674. This may allow the outer edge 670 of the cylindrical heating element 626 to come into contact with and vaporize a given quantity of phyto material extract 674. When compared to leaf phyto material that may be densely packed into an internal cavity of a heating chamber, the heating element 626 may come into more direct contact with the phyto material extract 674. Accordingly, the phyto material extract 674 may be more easily and consistently heated to its specific vaporization temperature.

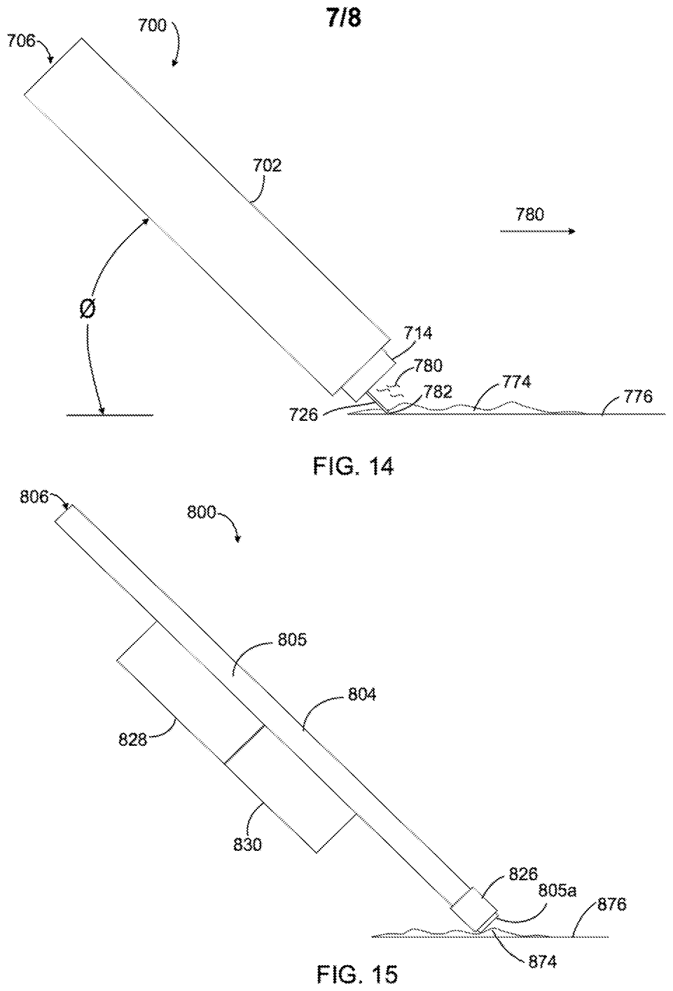

FIGS. 13 and 14 illustrate another example vaporization device 700 in accordance with an embodiment. Elements having similar structure and/or performing similar function as those in the example vaporization device 100 in FIGS. 1 to 2 are numbered similarly, with the reference numerals incremented by 600.

Vaporization device 700 is another example vaporization device that may be usable without loading phyto material extract into a heating chamber. Vaporization device includes a heating element 726 that extends outward from the housing 702. As shown, a flat plate heating element 726 is coupled to the first end 702A of the housing 702. The heating element 726 extends passed the housing first end 702A such that a portion of the heating element 726 protrudes from the housing first ends 702A.

In the illustrated example, an outer tip 782 of the flat plate ceramic heating element 726 extends a distance D_{y} from the housing first end 602A. As shown in FIG. 13, the internal cavity 724 of the heating chamber 714 may be in fluid communication with fluid pathway 704 through one or more vents 722 on the closed chamber wall 720. Vaporization device 700 is another example of a vaporization device in which the heating chamber 714 may operate as an inhalation or vapor gather chamber when the heating element 726 vaporizes phyto material extract external to vaporization device 700.

In use, the outer tip 782 of heating element 726 may be positioned to contact with phyto material extract 774 provided on a surface 776 (see e.g. FIG. 14). The vaporization device 700 may be used to vaporize the phyto material 774 in a similar manner as the vaporization device 600 illustrated in FIGS. 10 to 12 and with the similar predefined vaporization temperature.

As shown in FIGS. 13 and 14, the heating element 726 may be offset from a central axis 784 of the cylindrical heating chamber 714. For instance, the heating element 726 may be offset on the opposite of the central axis from the inhalation aperture 706. By positioning the inhalation aperture 706 offset to a first side of the central axis, a user may be encouraged to use the vaporization device with the inhalation aperture 706 at a raised or upper position. Thus, the heating element 726 may be positioned proximate the surface on which the phyto material 774 is positioned. By offsetting the heating element 726 from the vapor inlets, the vapor emitted from the phyto material 774 may be encouraged to flow upwards into the vapor inlets 722.

The vaporization devices shown in FIGS. 10-15 may also be usable as a wand that may be used to poke, stir or to heat phyto material or phyto material extract disposed within bowl or other non-planar surface.

FIG. 15 illustrates another example vaporization device 800 in accordance with an embodiment. Elements having similar structure and/or performing similar function as those in the example vaporization device 100 in FIGS. 1 to 2 are numbered similarly, with the reference numerals incremented by 700.

The example vaporization device 800 is shown with its housing removed. As shown in FIG. 15, the vaporization device 800 may include a tube 805 enclosing a continuous fluid pathway 804. An energy storage member 828 and control circuit 830 may be mounted to one side of the fluid pathway 804. A tube 805 forming the fluid pathway 804 may be manufactured of glass. For example, the tube 805 may be manufactured using a fused quartz glass or a borosilicate glass.

The fluid pathway 804 may extend between an inhalation aperture 806 and a heating element 826. The heating element 826 may be mounted at, or proximate to, the first end 805a of the tube 805. The heating element 826 may heat the tube 805, to the predefined vaporization temperature, which in turn may be brought into contact with the phyto material extract 874. The tube 805, thus heated may be used in a manner analogous to those of vaporization devices 600 and 700 to vaporize phyto material extract 874 positioned on a surface 876.

In some cases, the heating element 826 may be slightly offset from the first end 805a. This may ensure that the heating element 826 does not directly contact the phyto material 874 being vaporized. Rather, the heating element 826 heats the glass tube 802 through conductive heating and then the tube 805 heats the phyto material extract when placed in contact therewith.

The tube 805, heating element 826, energy storage member 828 and control circuit 830 may be enclosed within a housing such as those described herein above. The first end 805a of the tube 805 may protrude from the housing to allow the heated tube 805 to contact phyto material extract directly. Accordingly, the vaporization device may include a heating chamber operable as a vapor gathering chamber in such cases. Using a glass tube 802 may provide for a cleaner experience when inhaling of vapor derived from the phyto material extracts.

In this embodiment it may be preferable to have the user-activated switch in order to facilitate heating of the heating element 826 to a predefined vaporization temperature of about 632 degrees Fahrenheit and then to provide a notification to the user when that temperature has been approximately achieved.

FIG. 16 illustrates another example vaporization device 900 in accordance with an embodiment. Elements having similar structure and/or performing similar function as those in the example vaporization device 100 in FIGS. 1 to 2 are numbered similarly, with the reference numerals incremented by 800.

As shown in FIG. 16, vaporization device 900 includes a housing 902 that extends from an inhalation aperture 906 to a heating chamber 914. A heating element 926 protrudes from the first end 914A of the heating chamber 914. In vaporization device 900, the heating chamber 914 may operate as a vapor gathering chamber when heating element 926 is used to vaporize phyto material or extract external to vaporization device 900.

The heating element 926 may include an electric coil enclosed within a heating element housing 927. The coil may be a resistive wire coil operable to radiate heat outwards towards the heating element housing 927. The heating element housing 927 may then contact and vaporize phyto material extract.

As explained above, using a wire coil, or resistance wire wrapped electrical coil, heater may provide for more rapid heating times. Accordingly, in some embodiments, the heating element 926 may be activated in response to the detection of a user inhaling from inhalation aperture 906 (or an adjacent secondary inhalation aperture). In the case when the heating element may be activated in response to the detection of a user inhaling from inhalation aperture then the heating element 926 and preferably the heating element housing 927 may be manufactured from a low thermal inertia to facilitate quick heating of about a second or so. In some embodiments it may be preferable to have a user-activated switch in order to facilitate heating of the heating element 926 and preferably the heating element housing 927 the predefined vaporization temperature of about 632 degrees Fahrenheit (or to within an adjustable range of about 500 Fahrenheit to about 700 Fahrenheit) and then to provide a notification to the user when that temperature has been approximately achieved.

The heating element housing 927 may facilitate cleaning of the vaporization device 900. For instance, the heating element housing 927 may be manufactured using glass or another relatively easily cleanable material that is non-porous. Thus, the heating element housing 927 may be wiped clean by a user after use, while protecting the coil heater from becoming dirty or clogged. In some embodiments the heating element housing 927 may be manufactured from a porous ceramic material that may wick in the phyto material extract directly and hold the material within the porous ceramic material for heating by the heating element.

FIG. 17 illustrates another example vaporization device 1000 in accordance with an embodiment. Elements having similar structure and/or performing similar function as those in the example vaporization device 100 in FIGS. 1 to 2 are numbered similarly, with the reference numerals incremented by 900.

As shown in FIG. 17, vaporization device 1000 includes a housing 1002 that extends from an inhalation aperture 1006 to a heating chamber 1014. A heating element 1026 protrudes from the first end 1014A of the heating chamber 1014. In vaporization device 1000, the heating chamber 1014 may operate as a vapor gathering chamber when the protruding heating element 1026 is used to heat phyto material or extract external to the heating chamber 1014.

The heating element 1026 includes may include an electric coil mounted on a heating element support 1029. For instance, the heating element support 1029 may be a quartz or ceramic rod extending from the second end of the heating chamber 1014.

As with heating element 926, the coil may be a resistive wire coil operable to radiate heat. The heating element 1026 may be exposed to directly contact phyto material extract. Accordingly, the heating element 1026 may contact and vaporize phyto material extract directly.

As explained above, using a wire coil heater may provide for more rapid heating times. Accordingly, in some embodiments, the heating element 1026 may be activated in response to the detection of a user inhaling from inhalation aperture 1006 (or an adjacent secondary inhalation aperture). Providing an exposed heating element 1026 that may directly contact the phyto material or extract may further reduce the ramp-up time to achieve the vaporization temperature. In some embodiments an electrical user activated switch may be utilized in order to control electrical power being provided to the heating element.

The embodiments described for FIGS. 1 through 17 may be for use with phyto materials and for phyto material extracts as described herein. However, in these embodiments illustrate for FIGS. for FIGS. 1 through 17, the vaporization device is for use with an external source of phyto material or phyto material extract. Meaning that the used may hold a container that contains phyto material extract or phyto material external to the vaporization device. In such embodiments it may require two handed operation or use with the help of another surface in order to facilitate vaporization of the phyto material or phyto material extracts. For example, the phyto material is disposed on a surface and the vaporization device is brought within close proximity to the surface. In other cases the phyto material or phyto material extract may be contained within a container and the vaporization device may be brought within close proximity to the phyto material extract or phyto material contained within the container and the material is the vaporized by the vaporization device. It may be preferable and it may be advantageous to provide for a means of utilizing the vaporization device in a single-handed manner or operation without requiring a surface external to the vaporization device for containing of the phyto material or phyto material extract.

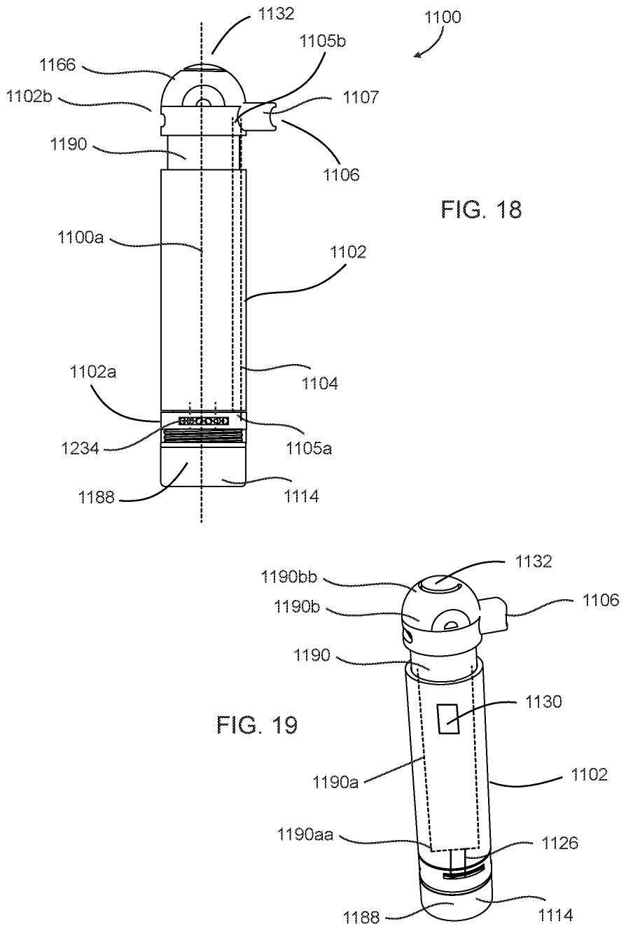

As shown in FIGS. 18 to 22, a vaporization device 1100, in accordance with embodiments of the invention, that facilitates single handed operation use, will be descried hereinbelow. The vaporization device 1100 is shown from a side view that includes a housing 1102 that extends from an inhalation aperture 1106 to a removable heating chamber 1114 or removable storage container 1188. The vaporization device 1100 may be formed from the housing 1102 having a longitudinal axis 1100a disposed axially from and a housing first end 1102a to a second housing 1102b end opposite the housing first end 1102a.

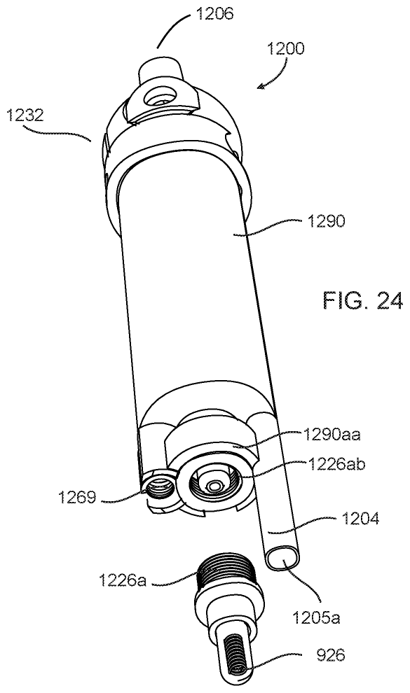

A slider assembly 1190 for sliding parallel with the longitudinal axis 1100a and in relation with the housing 1102 is provided with a vapor conduit or fluid pathway 1104 extending axially between a first open conduit end 1105a and a second open conduit end 1105b, the vapor conduit having an outer conduit surface enclosing the fluid pathway 1104 extending between the first open conduit end 1105a and the second open conduit end 1105b, wherein the second open conduit end 1105b defining an inhalation aperture 1106. The heating element 1126 may have a releasable coupling 1126a, such as a threaded coupling (see FIG. 24) or a magnetic coupling or a frictionally coupling with a heating element receptacle (FIG. 27 1126ab) where a used or worn heating element is easily replaced. There may be electrical energy transferred through the releasable coupling 1126a between the heating element 1126 and the receptacle 1126ab. In some embodiments the slider assembly 1190 may be coaxial with the housing 1102 with longitudinal axis at a center thereof.

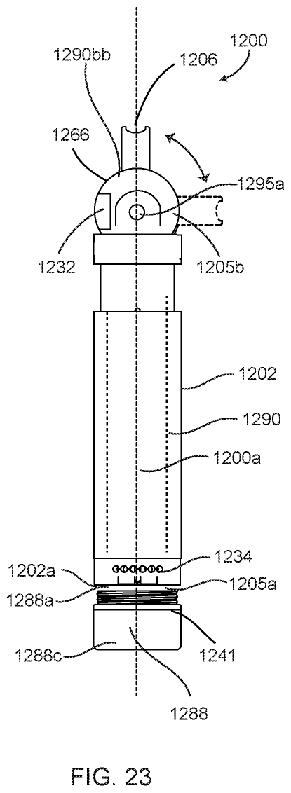

A removable heating chamber 1114 or removable storage container 1188 bounded by at least two sides and having an open end 1188a for receiving and for storing of phyto material extract, the removable storage container open end 1188a may be for engaging with the housing first end 1102a for forming a substantially enclosed heating chamber 1114 when the removable storage container 1188 may be coupled with the housing 1102. An ambient air input port 1134 fluidly coupled with the removable heating chamber 1114 for allowing ambient air 555 to enter into the substantially enclosed removable heating chamber 1114 when the removable heating chamber is coupled with the housing 1102, as shown in FIG. 23. When the removable storage container 1188 is other than coupled with the housing 1102 it may facilitate of loading of phyto material extracts there.

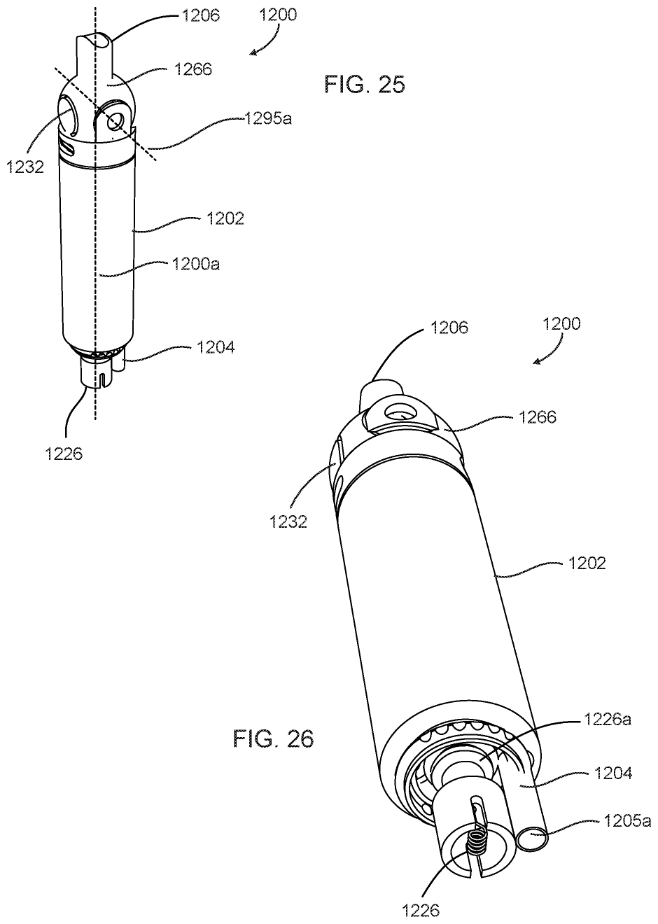

FIGS. 25 and 26 illustrates the slider assembly 1190 with the heating element 1126 being coupled with the slider assembly 1190 and in the second mode of operation where the heating element assembly is exposed past the housing first end 1102a where a first portion of the slider assembly 1190, a slider assembly first portion 1190a is disposed within the housing 1102 (as shown) and a second potion of the slider assembly 1102, a slider assembly second portion 1190b extends beyond the housing 1102 second end 1102b. The slider assembly 1190 may include a first end 1190aa is proximate the first open conduit end 1105a and proximate the housing first end 1102a, the heating element 1126 operable to heat to a predetermined vaporization temperature (such as between 500 degrees Fahrenheit to about 800 degrees Fahrenheit) wherein in use the predetermined vaporization temperature is sufficient to vaporize phyto material extract 774 in contact with the heating element 1126 that is heated to the predetermined vaporization temperature. An energy storage member 1128, such as a rechargeable lithium battery, is electrically coupled to the heating element 1126 and a control circuit 1130. The control circuit 1130 operable to control a flow of electric current from the energy storage member 1128 to the heating element 1126.

In response to a user depressing on the slider assembly 1190 and transitioning the vaporization device 1100 from the first mode of operation to the second mode, or prior to the user depressing on the slider assembly 1190, the control circuit 1130 may be engaged for electric current to flow from the energy storage member 1128 to the heating element 1126 for heating of the heating element 1126 to the predefined vaporization temperature and for the heating element 1126 sliding away from the first end of the housing 1102a towards the phyto material extract 774 and for being at least partially inserted into the removable storage container 1188 or removable heating chamber 1114 and the heating element 1127 for contacting the phyto material extract 774 and for creating a phyto material extract vapor to be formed within the removable heating chamber 1114, which is the second mode of operation.

Upon the user providing an inhalation at the inhalation aperture 1106, a pressure gradient is created across the fluid pathway 1104 that draws ambient air 555 from the ambient air input port 1134 from the external environment into first open conduit end 1105a and the ambient air 555 mixes with the phyto material extract vapor, and the mixed vapor and air are drawn through the fluid pathway 1104 to the inhalation aperture 1106.

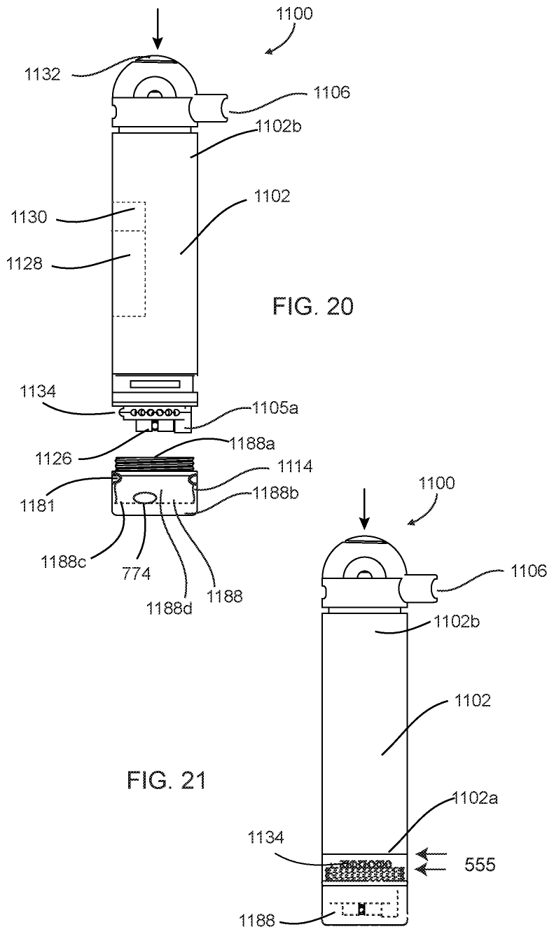

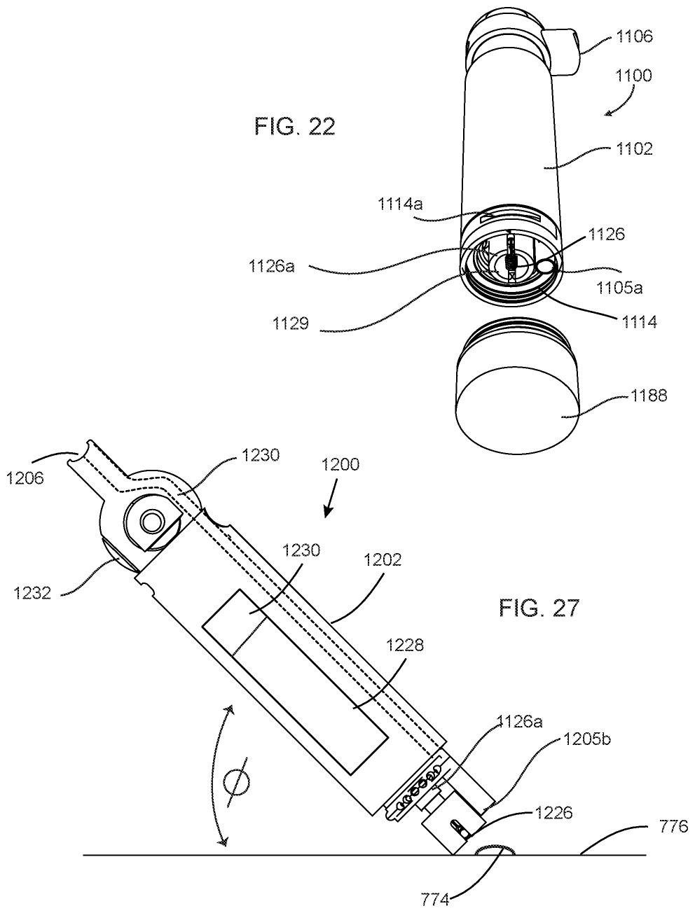

The heating element 1126 and slider assembly 1190 operate in a first mode of operation where is at least partially disposed within the housing as shown in FIG. 22 and in the second mode the heating element 1126 protrudes past the housing first end 1102a into the removable heating chamber 1114 as shown in FIG. 20.

The removable storage container 1188 may be formed from a first end 1188a and a second end 1188b opposite the first end 1188a and one or more sidewalls extending from the first end to the second end and a third sidewall capping the removable storage container 1188 proximate the second end, the one or more sidewalls together with the third sidewall defining the removable storage container 1188 a storage chamber cavity having the first open 1188a end proximate the housing first end 1102a when the removable storage container 1188 may be coupled with the housing. When the removable storage container 1188 is other than coupled with the housing 1102, for the phyto material extract 774 to be inserted into the storage chamber cavity 1188c.

The phyto material extract 774 that may be disposed within the removable storage container 1188 and more specifically within the storage chamber cavity 1188c may be substantially contained within a removable storage container 1188 that is removably coupled (ie. threaded, or frictionally engaged or magnetically coupled) with the housing 1102.

There may be adjustable ambient air airflow vents 1187 that are provided for adjustably controlling the ambient air 555 that enters in the removable heating chamber 1114, these air vents may be adjustable by the user to control an amount of ambient air that mixes with vapor (i.e. a rotating collar or a slider is envisaged to open and close vent apertures to expose more apertures to incoming ambient air). There may be a single aperture and there may be a plurality of these apertures for the ambient air input port 1134.

In some embodiments the heating element 1126 may be disposed adjacent the first open conduit end 1105a (as shown in the embodiment illustrated in FIGS. 18 to 22), in other embodiments the first open conduit end 1105a may be formed using a tube 1105 that may be heated may in a manner analogous to those of vaporization devices 600 and 700 to vaporize phyto material extract 774 positioned within the container 1188. The heating element 1126 may heat the phyto material proximate the tube 1105 which in turn may be brought into contact with the phyto material 774 where the tube 1105 may have a thick film heater or heating element formed proximate the first open conduit end 1105a.

The heating element and the mouthpiece may be slidably coupled within the housing on the slider assembly 1190 such that when the slider assembly is depressed proximate the second end 1102b of the housing the heating element 1126 extends pass the housing first end 1102a such that a portion of the heating element 1126 protrudes from the housing first ends 1102A and may protrude into the container 1188. The removable storage container may include a floor and walls where the floor may be covered with phyto material or phyto material extract and upon the slider being depressed proximate the second end the heating element contacts the phyto material or phyto material extract and upon the heating element being energized, phyto material in contact therewith and or proximate thereto through convective heating will also vaporize or at least partially vaporize depending on the temperature of the heating element.

Preferably a user-activated switch 1132 (i.e. a tactile switch or a capacitive switch) is found proximate the second end 1190bb of slider assembly 1190 and mounted to the slider assembly 1190 such that when the slider assembly is pressed by the user the switch may simultaneously be activated through the pressing force and it may cause heating of the heating element and the heating element being heated as the slider assembly slides within the housing 1102 so that when the heating element is inserted into the phyto material extract it is already hot (i.e. at the predetermined vaporization temperature) and vaporizes the phyto material extract. Proximate the second end 1190bb of slider assembly 1190 the user-activated switch 1132 as well as the inhalation aperture 1106 may form a slider assembly head 1166. The inhalation aperture 1106 being oriented such that it is approximately perpendicular with the longitudinal axis 1100a. In such an orientation, when for example the user holds the vaporization device 1100 with their right hand, where fingers are wrapped around the housing 1102 and a thump of the user contacts the slider assembly head 1166. Then the inhalation aperture 1106 protrudes from their palm proximate the thumb. This allows for the user's fingers to firmly grasp the housing 1102 of the vaporization device 1100 and for the thumb to be used to actuate the user-activated switch 1132 as well as for the thumb to actuate the slider assembly 1190 for moving the heating element towards the phyto material extract. In such an embodiment the slider assembly 1190 is for sliding within the housing 1102 and parallel with the longitudinal axis 1100a and the vapor conduit or fluid pathway 1104 is formed as part of the slider assembly 1190. Through sliding of the slider assembly 1190 within the housing 1102, this facilitates the user for holding of the housing 1102 with approximately 4 of their fingers (other than the thumb) and for the housing 1102 to rest in a palm of their hand. Preferably the vaporization device is palm sized for being easy to conceal and to be portable.

In vaporization device 1100, the removable heating chamber 1114 may operate as a vapor gathering chamber and a vapor cooling chamber when the protruding heating element 1126 is used to heat phyto material extract contained within the removable heating chamber 1114. Proximate the mouthpiece there may also be a vapor gathering and cooling chamber 1107 that may be formed within the slider assembly head 1166 that may be fluidly coupled to the vapor conduit 1104 for providing of further cooling of the vapor and ambient air mixture.

In some embodiments the slider assembly 1190 may be spring loaded using a spring (not shown) so that after the slider assembly 1190 is depressed and slides towards and into the removable storage container 1188, the spring may be used to retract the heating element 1126 from within the removable storage container 1188 to its resting state or resting position in the first mode of operation, whereby it is not at least partially extended past the housing.

The user overcomes a spring force of the spring so that a force is exerted to extend the heating element and the spring force retracts the heating element. Optionally an elastomeric force or a magnet force is also envisaged. The inhalation tube may be mounted with the housing or with the slider assembly. The housing and the slider mechanism may be about 20 mm in diameter and may be about 130 mm long and may be fit into a hand and are preferably able to be activated using a single movement of the hand such that the housing rests in the palm of a user and the slider assembly head 1166 or top of the slider assembly 1190bb is depressed using a thumb of the user. In some embodiments the switch 1132 may be depressed first by the user and a notification may be provided to the user as to when to press further on the or top of the slider mechanism 1190bb in order to further press the heating element into the removable storage container 1188 and for contacting the phyto material extract 774.

By controlling a time when the switch 1132 is depressed and a rate at which the slider assembly slides along with the heating element sliding in relation to the housing towards the phyto material extract, a user is able to control a vaporization temperature through titration and a length of time and insertion depth when the heating element 1126 is put into contact with the phyto material extract stored within the container 1188. The user may experiment and determine what works best for their preferences, however the user is able to actuate the slider assembly with their thumb while holding onto the housing so that they are able to controllably provide more or less vapor to be emitted from the inhalation aperture as needed.

Preferably the heating element is prevented from pressing into a bottom surface, or the third sidewall defining the removable storage container 1188 of the storage chamber cavity or the third surface, of the container so that thermal damage to the container does not take place of the storage chamber cavity. In some embodiments the storage chamber cavity is sloped so that a well is formed within the storage chamber cavity proximate where the heating element 1126 is closest to the third sidewall 1188c so that when the slider assembly 1126 is fully extended past the housing 1102 the heating element is at a distance from the third sidewall of the storage chamber cavity, for example 1 mm or 1.5 mm. In some embodiments removable storage container 1188 is formed from a glass or ceramic or metal material. Preferably other than plastic material is so that the removable storage container 1188 does not melt in proximity of the heating element 1126. In some embodiments the housing 1102 is adapted to work with off the shelf phyto material storage containers either directly or with an additional coupling adapter.

Optionally a temperature setting is provided for the heating element so that a preset temperature is able to be set and the heating element at a predetermined temperature may be slid into the removable container. In some embodiments a temperature sensor is utilized in order to determine a temperature of the heating element, in other embodiments a pulse width modulation (PWM) heating profile is applied to the heating element for providing of a desired heating temperature over time. In some embodiments the user may be able to adjust the predetermined temperature using a user input, such as a button or resistor wheel or digital control for providing of a temperature setting signal to the control circuit.

Referring to FIG. 20, the removable storage container 1188 is illustrated being uncoupled from the housing 1102. Firstly, the user may load the phyto material into the cavity of the storage container 1188, the storage container may then be coupled with the housing as shown in FIG. 18. The storage container may include an external thread and the housing includes an internal thread and the inside of the storage container may be lined with a silicone material and the heating element extends into the storage container so that it does not contact the floor thereof so that it does not burn the silicone material, so a distance of about 5 mm is envisaged.

However, as stated above, it may be more preferable to manufacture the removable storage container from a non-thermoplastic material. Optionally the storage container is a glass storage container. An adapter ring is provided that couples the storage container with the housing so that a glass storage container having a larger diameter or a smaller diameter or a same diameter as that of the housing is provided for releasable coupling. In some embodiments the heating element is at least partially shrouded (see FIG. 22) where the heating element is partially recessed without a heating element housing 1126a so that the heating element may not protrude past the housing and allows for the housing to contact the phyto material extract first prior to the heating element to contact the phyto materiel extract.

Optionally an internal rib 1181 is provided as part of the storage container 1188 on the cavity side with the internal rib 1181 extending radially towards the longitudinal axis 1100a when the removable storage contained is coupled with the housing. The internal rib 1181 facilitates to contain a substantial amount or at least some of the phyto material extract when the device for vaporization 1100 is rested on its side (i.e. when the longitudinal axis is approximately parallel with a contacting planar surface and gravity is acting approximately equally along the longitudinal axis). A diameter of an internal rib 1181 may be sized so that it facilitates penetration of the heating element therein and a small gap of about 0.5 mm is formed about the heating element so that phyto material extract is contained within the cavity and doesn't substantially leak past the cavity into the fluid pathway 1104.

The heating element 1126 may include an electric coil mounted on a heating element support 1129 (FIG. 22). For instance, the heating element support 1129 may be a quartz or ceramic rod extending from the second end of the heating chamber 1114. The heating element may be a ceramic flat plate heater or the heating element may be a resistive coil or the heating element may be a resistive coil encased in a glass tip.

The heating element 1126 may also be formed using a laser diode with a short focus lens assembly (around 20 mm to 25 mm) having a wavelength of less than 570 nm and around 2 W to 5 W in power and the laser diode may not operate when the container is not coupled with the housing for safety reasons. For example, there may be a safety interlock between the container and the housing and the removable container may include a portion to complete circuitry so that the laser does not function without the container being attached. The container may contain a magnet and the housing include a hall effect switch. For example, a safety switch is used. In some embodiments the laser assembly may allow for the user to change a focal length of the lens such that the laser light may be focused on to the phyto material extract through the focusing operation or that the adjustment of the focusing of the lens of the laser light may obviate a need to slide of the slider assembly.

In some embodiments a 1500 mw blue laser (around 360 nm to 470 nm) may be used to heat the phyto material extract. Through pulse width modulation the laser may be controlled to control a heating of the phyto material extract. The laser light is enclosed in the housing and the removable container and preferably the housing and the container are non-laser wavelength transparent for the wavelength of the laser light so that the laser light does not shine past the housing to potentially harm the user. A focal length of the laser may be adjustable so that a larger area of the phyto material extract may be exposed to the light or more focused for a tighter area. In some embodiments an amount of heat that is imparted to the phyto material by the laser light may be dependent on an absorption of the laser light by the phyto material extract and in some cases darker colored phyto material extracts may absorb more light than lighter colored ones and as such heat at a faster rate. In some cases using of laser light with leaf phyto material may also be envisaged. In some cases the power of the laser is adjustable by the user using a user input control.

Preferably the slider assembly may be removed from the housing so that it allows for cleaning around the heating element receptacle. Electrical contacts may be provided from the control circuit 1130 to the heating element using the heating element receptacle (1226a). In some embodiments the heating element may be partially shrouded so as to not touch the third sidewall of the removable storage container cavity.

FIG. 20 shows the heating element protruding past the housing when the slider assembly is depressed and the removable storage container is uncoupled from the housing. FIG. 21 shows the storage container coupled with the housing and with the with the heating element protruding past the housing. FIG. 22 shows the vaporization device 1100 from a perspective view with the container uncoupled from the housing and the heating element 1126 visible as well as the heating chamber shown 1114.

As with heating element 926, the coil may be a resistive wire coil operable to radiate heat. The heating element 1026 may be exposed to directly contact phyto material extract. Accordingly, the heating element 1026 may contact and vaporize phyto material extract directly. The heating element may also be a shroud and the shroud may comprise a glass or quartz or stainless steel or other material so that thermal energy from the resistive heating element radiates through the shroud for then further contact with the phyto material extract.

Through detachment of the removable storage container 1188 the heating element is exposed and able to be removed and cleaned and the storage container may be replenished with phyto material extract as needed. In some embodiments the storage container includes tapered walls that allow for the phyto material extract to flow towards a center thereof so that as the phyto material extract is vaporized by the heating element, neighboring material has its viscosity altered and it flows towards a center thereon.

Accordingly, in some embodiments, the heating element 1126 may be activated in response to the detection of a user depressing the slider assembly for sliding of the heating element into the phyto material extracts. Providing an exposed heating element 1126 that may directly contact the phyto material or extract may further reduce the ramp-up time to achieve the vaporization temperature.

Referring to FIGS. 23 to 26 embodiments of the invention are shown similar to those of embodiments shown in FIGS. 18 to 22. Elements having similar structure and/or performing similar function as those in the example vaporization device 1200 in FIGS. 23 to 26 are numbered similarly, with the reference numerals incremented by 100. The embodiments shown in FIGS. 23 to 26 embodiments may be transformable with the use of a pivotable head 1295 as part of a slider assembly 1290. The slider assembly head 1266 or top of the slider assembly 1290 may be pivotable about a transverse axis 1295a that may be approximately perpendicular to a longitudinal axis 1200a. As shown in FIGS. 23 and 25, the vaporization device 1200 may be formed from a housing 1202 having the longitudinal axis 1200a disposed axially from and a housing first end 1202a to a second housing 1202b end opposite the housing first end 1202a. The pivotable head 1295 may be able to be approximately oriented with the inhalation aperture 1206 approximately perpendicular with the longitudinal axis 1200a (a first orientation) and or the inhalation aperture 1206 to be axial (or parallel) with the longitudinal axis 1200a (a second orientation), where a pivot angle of approximately forty five to about eighty nine degrees may be envisaged.

The pivotable head 1295 preferably may be frictionally movable in relation to the slider assembly 1290 where once it is oriented it remains in that orientation until re oriented or repositioned. In some embodiments the slider assembly may be for sliding within the housing along the longitudinal axis, which is coaxial to both the housing 1202 and the slider assembly 1290.

In some embodiments the pivotable head 1295 may be coupled with the slider assembly 1290 proximate the second end 1202b of the housing 1202 and for extending past the housing second end 1202b, the pivotable head 1295 may be for being pivotable about the transverse axis 1295a that may be approximately perpendicular to the longitudinal axis 1200a with the pivotable head 1295 in a first orientation with the inhalation aperture 1206 oriented radially from the coaxial longitudinal axis 1200a and in the second orientation the inhalation aperture 1206 oriented coaxially with the longitudinal axis 1200a that is coaxial to both the with the slider assembly 1290 and the housing 1202.

Referring to FIG. 23, the vaporization device 1200 is shown from a side view and in FIG. 25 from a top perspective view. Referring to FIGS. 23 and 25, the vaporization device includes a housing 1202 that may extend from an inhalation aperture 1206 to a removable heating chamber 1214 or removable storage container 1288. The slider assembly 1290 may be for sliding parallel with the longitudinal axis 1200a and in relation with the housing 1202 is provided with a vapor conduit or fluid pathway 1204 (FIG. 24 and FIG. 26) extending axially between a first open conduit end 1205a and a second open conduit end 1205b, the vapor conduit 1204 having an outer conduit surface enclosing the fluid pathway 1204 extending between the first open conduit end 1205a and the second open conduit end 1205b, wherein the second open conduit end 1205b may define an inhalation aperture 1206.

Through the pivoting of the slider assembly 1290, the vapor conduit 1204 may remain sealed with the inhalation aperture 1206 through elastomeric sealing such that when upon the user providing an inhalation at the inhalation aperture 1206, a pressure gradient is created across the fluid pathway 1204 that draws ambient air from the ambient an input port 1234 from the external environment into first open conduit end 1205a and the ambient air mixes with the phyto material extract vapor, and the mixed vapor and air may be drawn through the fluid pathway 1204 to the inhalation aperture 1206. In some embodiments a line coincident with the longitudinal axis 1200a of the may form a central plane positioned to contain a line coincident with the longitudinal axis, wherein the fluid pathway first end 1205a may be offset from the central plane and the vapor conduit 1204 and the inhalation 1206 aperture may be also offset from the central plane.

A removable heating chamber 1214 or removable storage container 1288 bounded by at least two sides and having an open end for receiving and for storing of phyto material extract, the removable storage container open end 1288a may be for engaging with the housing first end 1202a for forming a substantially enclosed heating chamber when the storage removable container 1288 may be coupled with the housing 1202 (see FIG. 18 for similar construction). The ambient air input port 1234 may be fluidly coupled with the removable heating chamber 1214 for allowing ambient air to enter into the substantially enclosed removable heating chamber 1214 when the removable heating chamber is coupled with the housing 1202 (FIG. 23 shows the removable storage container 1288 being uncoupled, for a coupled view refer to FIG. 18).

FIG. 26 illustrates a heating element 1226 may be coupled with the slider assembly 1290 and FIG. 24 illustrates the heating element and in this case heating element heating element 926 (such as that of FIG. 16) may be uncoupled with the slider assembly 1290. The slider assembly 1290 includes a first end 1290aa is proximate the first open conduit end 1205a and proximate the housing first end 1202a. The heating element 1226 may include a releasable coupling, such as a threaded coupling 1226a (see FIG. 24) or a magnetic coupling or a frictionally coupling with a heating element receptacle (FIG. 27 1226ab) where a used or worn heating element is easily replaced. There may be electrical energy transferred through the releasable coupling between the heating element 1226 and the receptacle 1226ab.

When the pivotable head 1295 may be in the first orientation then the vaporization device 1200 operates similarly with that shown and described in FIGS. 18 to 22, where the device may be usable in the first and second modes of operation.

In the first orientation of the pivotable head 1295, in response to a user depressing on the slider assembly 1290 and transitioning the vaporization device 1200 from the first mode of operation to the second mode, or prior to the user depressing on the slider assembly 1290, a control circuit 1230 (FIG. 27) is engaged for electric current to flow from an energy storage member 1228 (FIG. 27) to the heating element 1226 for heating of the heating element 1226 to the predefined vaporization temperature and for the heating element 1226 sliding away from the first end of the housing 1202a towards the phyto material extract and for being at least partially inserted into the removable storage container 1288 or removable heating chamber 1214 and the heating element 1226 for contacting the phyto material extract and for creating a phyto material extract vapor to be formed within the removable heating chamber 1214 such that upon the user providing an inhalation at the inhalation aperture 1206, a pressure gradient may be created across the fluid pathway 1204 that draws ambient air 555 from the ambient air input port 1234 from the external environment into first open conduit end 1205a and the ambient air mixes with the phyto material extract vapor, and the mixed vapor and air are drawn through the fluid pathway 1204 to the inhalation aperture 1206.

In some embodiments the slider assembly 1290 may be spring loaded in relation to the housing 1202 using a spring assembly 1269 having a spring force so that after the slider assembly 1290 may be depressed and slides towards and into the removable storage container 1288, the spring may be used to retract the heating element 1226 from within the removable storage container 1288 to its resting state or resting position in the first mode of operation, whereby it may be not at least partially extended past the housing first and 1202a. The user may overcome the spring force of the spring so that a force may be exerted to extend the heating element and the spring force retracts the heating element.

FIG. 27 illustrates vaporization device 1200 when the pivotable head 1295 may be in the second orientation and the vaporization device is operating in the second mode of operation and the vaporization device 1200 operates similarly with that shown and described in FIGS. 16 to 17. In some embodiments when the pivotable head 1295 is in the second orientation, the slider assembly 1290 and the first end 1290aa may be exposed past the first end of the housing 1202a and preferably latched in place using a latching mechanism (not shown) so than the spring force may be overcome by the second orientation of the pivotable head 1295. Once the pivotable head 1295 may be transitioned from the second orientation to the first orientation the spring force may be overcome and the heating element 1226 may be retracted from protruding past the housing first end 1202a and to its resting state or resting position in the first mode of operation.

Referring to FIG. 27, in use of the vaporization device 1200 the pivotable head 1295 may be in the second orientation and may be the second mode of operation, upon the heating element being energized to the predetermined temperature for phyto material extracts, and the heating element 1226 may be positioned to contact with phyto material extract 774 provided on a surface 776, the vaporization device 1200 may be used to vaporize the phyto material extract 774 in a similar manner as the vaporization device 600 illustrated in FIGS. 10 to 12. The vaporized phyto material is then inhaled from the inhalation aperture 1206.

The phyto material extract 774 may be disposed within the removable storage container 1288 and more specifically within a storage chamber cavity 1288c and may be substantially contained within the removable storage container 1288 that may be removably coupled (ie. threaded, or frictionally engaged or magnetically coupled) with the housing 1202. There may be an ambient air input port 1234 for controllably adjustably a flow of ambient air that enters in the removable heating chamber 1241, these air vents may be adjustable by the user to control an amount of ambient air that mixes with vapor (i.e. a rotating collar or a slider is envisaged to open and close vent apertures to expose more apertures to incoming ambient air). There may be a single aperture and there may be a plurality of these apertures for the ambient air input port 1234.

The heating element 1226 and the inhalation aperture 1206 may be slidably coupled within the housing on the slider assembly 1290 such that when the slider assembly is depressed proximate the second end 1202b of the housing the heating element 1226 extends past the housing first end 1202a such that a portion of the heating element 1226 protrudes from the housing first end 1202A and may protrude into the container 1288. The removable storage container may include a floor and walls where the floor may be covered with phyto material or phyto material extract and upon the slider being depressed proximate the second end the heating element contacts the phyto material or phyto material extract and upon the heating element being energized, phyto material in contact therewith and or proximate thereto through convective heating will also vaporize or at least partially vaporize depending on the temperature of the heating element.

Preferably a user-activated switch 1232 (i.e. a tactile switch or a capacitive switch) may be found proximate the second end 1290bb of slider assembly 1290 and as part of the pivotable head 1295 and opposite the inhalation aperture 1206 and such that an orientation of the a user-activated switch 1232 in relation to the housing 1202 may be varied in dependence upon whether the pivotable head 1295 may be in the second orientation or may be the first orientation. In the first orientation of the pivotable head 1295 the user-activated switch 1232 may be activated along the longitudinal axis 1200a and in the second orientation the user-activated switch 1232 may be activated approximately perpendicular to the longitudinal axis 1200a.

In the first orientation pivotable head 1295 when the slider assembly 1290 is pressed by the user the a user-activated switch 1232 may be simultaneously activated through the pressing force and it may cause heating of the heating element 1226 and the heating element being heated as the slider assembly may slide within the housing 1202 so that when the heating element may be inserted into the phyto material extract it may be already at the predetermined vaporization temperature and vaporizes the phyto material extract.

In the second orientation pivotable head 1295 the a user-activated switch 1232 may be simultaneously activated through the pressing force and may causes heating of the heating element and the heating element is being heated so that when the heating element is inserted into the phyto material extract it may be already at the predetermined vaporization temperature and vaporizes the phyto material extract. In the second orientation of the pivotable head 1295 and the upon depressing of the user-activated switch 1232 the slider assembly may not slide within the housing 1202 because of the slider assembly 1290 may be preferably latched in place using a latching mechanism, such as a magnetic latching mechanism or a frictionally engaging latching mechanism (not shown) and the heating element 1226 is exposed past the first end of the housing 1202a.

In the first orientation of the pivotable head 1295 the user holds the vaporization device 1200, for example, with their right hand, where fingers are wrapped around the housing 1202 and the housing 1202 rests within the palm of the user's hand and a thump of the user contacts the slider assembly head 1266 and the user-activated switch 1232. Then the inhalation aperture 1206 may protrude from their palm proximate the thumb. This may allow for the user's fingers to firmly grasp the housing 1202 of the vaporization device 1200 and for the thumb to be used to actuate the user-activated switch 1232 as well as for the thumb to actuate the slider assembly 1290 for moving the heating element towards the phyto material extract. In such an embodiment the slider assembly 1290 may be for sliding within the housing 1202 and parallel with the longitudinal axis 1200a and the vapor conduit or fluid pathway 1204 may be formed as part of the slider assembly 1290. Through sliding of the slider assembly 1290 within the housing 1202, this may facilitate the user for holding of the housing 1202 with approximately 4 of their fingers (other than the thumb) and for the housing 1202 to rest in a palm of their hand.

In the second orientation of the pivotable head 1295 the user may hold the vaporization device 1200, for example, with their right hand, where fingers are holding the housing 1202 from a first side and a thumb of the user contacts the user-activated switch 1232 on a second side opposite to the first side.

By controlling a time when the switch 1232 is depressed and a rate at which the slider assembly slides along with the heating element sliding in relation to the housing towards the phyto material extract in the first orientation of the pivotable head 1295 or a rate at which the user inserts the heating element into the phyto material extract, in the second orientation of the pivotable head 1295, the user may be able to control a vaporization temperature through titration and a length of time and insertion depth when the heating element 1226 may put into contact with the phyto material extract. The user may experiment and determine what works best for their preferences, however the user may be able to actuate the slider assembly with their thumb while holding onto the housing so that they are able to controllably provide more or less vapor to be emitted from the inhalation aperture as needed or dipping of the heating element into the phyto material extract to control the amount of vapor.

Preferably the slider assembly 1290 may be removed from the housing 1202 so that may allow for cleaning around the heating element receptacle 1226ab. Electrical contacts may be provided from the control circuit 1230 to the heating element using the heating element receptacle (1226ab). In some embodiments the heating element may be partially shrouded so as to not touch the third sidewall of the removable storage container cavity and allow for phyto material extract for contacting of the heating element. Or in some cases a depth of travel within the removable storage container is fixed so that the heating element may not contact the removable storage container cavity floor.

As with heating element 926, the coil may be a resistive wire coil operable to radiate heat. The heating element 1026 may be exposed to directly contact phyto material extract. Accordingly, the heating element 1026 may contact and vaporize phyto material extract directly. The heating element may also be a shroud and the shroud may comprise a glass or quartz or stainless steel or other material so that thermal energy from the resistive heating element radiates through the shroud for then further contact with the phyto material extract.

Through detachment of the removable storage container 1288 the heating element may be exposed and able to be removed and cleaned and the storage container may be replenished with phyto material extract as needed. In some cases the storage container includes tapered walls as the internal cavity to allow for the phyto material extract to flow towards a center thereof (towards the axial longitudinal axis) so that as the phyto material extract is vaporized by the heating element, neighboring material has its viscosity altered and it flows towards a center thereof.

As explained above, using a wire coil heater may provide for more rapid heating times. Accordingly, in some embodiments, the heating element 1226 may be activated in response to the detection of a user inhaling from inhalation aperture 1206 (or an adjacent secondary inhalation aperture). Providing an exposed heating element 1226 that may directly contact the phyto material or extract may further reduce the ramp-up time to achieve the vaporization temperature.

In some embodiments a syringe made from glass that is known in the art and that is used to house phyto material extracts may be used as the removable storage container where a tip of the syringe is pointed towards the heating element and the syringe is positioned and held in a mount such that when the slider assembly is activated and the user pushes the heating element towards the removable storage container, which is the syringe, the heating element may be brought into proximity of the syringe tip and the heating element may heats the tip and the phyto material extract inside the syringe proximate the tip may be vaporized. The user may also push a plunger of the syringe to move additional phyto material extract therein towards the tip to increase an amount of phyto material extract being vaporized. The plunger of the syringe may also be disposed on a ratchet mechanism to control an amount of phyto material extract being dispensed towards the heating element.

In some embodiments the inhalation aperture may be coupled with a water filtration device or an ultrasonic misting device in order to water filter or moisturize or cool vapor being emitted from the inhalation aperture.

For facilitating single handed operation, preferably the vaporization device shown in FIGS. 20 to 27 may be about 12 cm to 15 cm in length along the longitudinal axis and about may be about 1.5 to 2 cm in diameter perpendicular with the longitudinal axis. Such a size may facilitate the user for holding of the housing 1202 with approximately 4 of their fingers (other than the thumb) and for the housing 1202 to rest in a palm of their hand and the thumb to be used to contact the head of the slider assembly.

In some embodiments, the heating element may be detachably attached to the vaporization device. This may facilitate cleaning of the heating element, particularly in embodiments in which the heating element contacts phyto material or extract directly (such as those shown in FIGS. 1, 6, 8-9, 13, 14, 16, and 17, 22-25, 18-20 for example). This may also facilitate replacement of the heating elements in case of damage.

In some embodiments, the vaporization devices may be convertible between an extract vaporization device and a phyto material vaporization device. For example, the vaporization devices 600 and 800 may be used to vaporize phyto material or phyto material extract by adjusting the predetermined vaporization temperature to a suitable temperature. Additionally, the vaporization devices 600/800 may vaporize external phyto material extract (or phyto material), while also enabling phyto material (or extract) to be positioned within the heating chamber for vaporization.

In some embodiments, the vaporization devices may include separate heating elements for different operational modes. For instance, vaporization devices 700, 900 and 1000 may include a secondary heating element positioned to contact the chamber wall of the heating chamber. This may allow the vaporization devices 700, 900, and 1000 to vaporize phyto material or extract positioned within the heating chamber even if the protruding heating element is removed. For the vaporization devices of 1100 and 1200, various heating elements may be used as shown in the vaporization devices 700, 900, 1000, 1100 and 1200.

As used herein, the wording “and/or” is intended to represent an inclusive—or. That is, “X and/or Y” is intended to mean X or Y or both, for example. As a further example, “X, Y, and/or Z” is intended to mean X or Y or Z or any combination thereof.

While the above description describes features of example embodiments, it will be appreciated that some features and/or functions of the described embodiments are susceptible to modification without departing from the spirit and principles of operation of the described embodiments. For example, the various characteristics which are described by means of the represented embodiments or examples may be selectively combined with each other. Accordingly, what has been described above is intended to be illustrative of the claimed concept and non-limiting. It will be understood by persons skilled in the art that other variants and modifications may be made without departing from the scope of the invention as defined in the claims appended hereto. The scope of the claims should not be limited by the preferred embodiments and examples, but should be given the broadest interpretation consistent with the description as a whole.

What I claim is:

1. A vaporization device comprising:
a housing having a longitudinal axis and a housing first end and a housing second end opposite the housing first end;
a slider assembly for sliding parallel with the longitudinal axis and in relation with the housing;
a vapor conduit extending axially between a first open conduit end and a second open conduit end, the vapor conduit having an outer conduit surface enclosing a fluid pathway extending between the first open conduit end and the second open conduit end, wherein the second open conduit end defining an inhalation aperture;
a removable storage container bounded by at least two sidewalls and having an open end forming a storage cavity for receiving and for storing of phyto material extract, the removable storage container open end for engaging with the housing first end for forming a substantially enclosed heating chamber when the storage container is coupled with the housing;
an ambient air input port fluidly coupled with the heating chamber for allowing ambient air to enter into the substantially enclosed heating chamber;
a heating element coupled with the slider assembly and proximate the first open conduit end and proximate the housing first end, the heating element operable to heat to a predetermined vaporization temperature wherein in use the predetermined vaporization temperature is sufficient to vaporize phyto material extract in contact with the heating element;
an energy storage member electrically coupled to the heating element; and
a control circuit electrically coupled to the energy storage member, the control circuit operable to control a flow of electric current from the energy storage member to the heating element,
the slider assembly and heating element biased with the heating element other than substantially extended from the housing in a first mode of operation;
wherein, in response to a user depressing on the slider assembly the control circuit is engaged for electric current to flow from the energy storage member to the heating element for heating of the heating element to the predefined vaporization temperature and for the heating element sliding away from the first end of the housing towards the phyto material extract and for being at least partially inserted into the storage container in a second mode of operation and the heating element for contacting the phyto material extract and for creating a phyto material extract vapor; and
where inhalation at the inhalation aperture, a pressure gradient is created across the fluid pathway that draws ambient air from the ambient air input port from the external environment into the first open conduit end and the ambient air mixes with the phyto material extract vapor and the mixed vapor and air are drawn through the fluid pathway to the inhalation aperture.

2. A vaporization device according to claim 1 comprising a spring having a spring force disposed between the slider assembly and the housing, wherein the slider assembly is biased by the spring with the force of the spring being overcome to operate from the first mode of operation to the second mode of operation.

3. A vaporization device according to claim 1 wherein the first housing comprises a central plane positioned to contain a line coincident with the longitudinal axis, wherein the fluid pathway first end is offset from the central plane and the heating chamber comprises the open end that is fluidly coupled with the fluid pathway, and the inhalation aperture is also offset from the central plane.

4. A vaporization device according to claim 3 wherein an energy storage member comprises a rechargeable battery that is electrically coupled to the heating element through the control circuit and the rechargeable battery is coaxial with the central axis.

5. A vaporization device according to claim 1 wherein the wherein the heating element is a blade-shaped heating element.

6. A vaporization device according to claim 1 wherein the heating element is a rod-shaped heating element.

7. A vaporization device according to claim 1 wherein the heating element is a cylindrical heating element.

8. A vaporization device according to claim 1 comprising a switch electrically coupled with the control circuit, the switch for being coupled with the slider assembly for being activated by the user when depressing on the slider assembly for providing a control signal to the control circuit for electric current to flow from the energy storage member to the heating element.

9. A herbal vaporization device comprising:
a housing comprising a first end, a second end disposed opposite the first end disposed coaxially with a slider assembly for sliding within the housing along a coaxial longitudinal axis;
a first battery at least partially disposed within the slider assembly;
a conductive heating element coupled with the slider assembly and disposed proximate the first end of the housing
a pivotable head coupled with the slider assembly proximate the second end of the housing and for extending past the housing second end, the pivotable head for being pivotable about a transverse axis that is approximately perpendicular to the coaxial longitudinal axis with the pivotable head being in a first orientation with an inhalation aperture oriented radially from the coaxial longitudinal axis and in the second orientation the inhalation aperture oriented approximately coaxially with the coaxial longitudinal axis;
a switch coupled with the pivotable head opposite the inhalation aperture;
a first control circuit coupled with the first battery and the conductive heating element and the switch, the first control circuit for controlling the flow of electrical current from the first battery to the conductive heating element, the switch coupled with the first control circuit for providing a control signal to the first control circuit for affecting the flow of electrical current from the first battery to the conductive heating element for heating of the conductive heating element to the predetermined vaporization temperature;
a fluid pathway extending through the slider assembly from the first end of the housing to past the second end of the housing the fluid pathway being fluidly coupled proximate the conductive heating element and terminating at the inhalation aperture;
wherein in a first orientation of the pivotable head the conductive heating element is retracted within the housing and upon depressing of the switch, the slider assembly is slid within the housing and the conductive heating element is extended from the housing and heated to the predetermined vaporization temperature in a second orientation of the pivotable head the conductive heating element is extended from the housing and upon depressing of the switch is heated to the predetermined vaporization temperature
the heated conductive heating element for contacting the phyto material extract and where upon creating a low pressure proximate the second end of the fluid pathway, a low pressure is created in the fluid pathway and ambient air at a higher pressure flows and mixes with vapor emitted from conductive heating of the phyto material extract and with the ambient air and together flows out of the inhalation aperture.

10. A vaporization device according to claim 9 wherein the first housing comprises a central plane positioned to contain a line coincident with the longitudinal axis, wherein the fluid pathway first end is offset from the central plane and the heating chamber comprises the open end that is fluidly coupled with the fluid pathway and the inhalation aperture is also offset from the central plane.

11. A vaporization device according to claim 9 wherein the wherein the heating element comprises a blade-shaped heating element.

12. A vaporization device according to claim 9 wherein the heating element comprises a rod-shaped heating element.

13. A vaporization device according to claim 9 wherein the heating element comprises a cylindrical heating element.

14. A vaporization device according to claim 9 comprising a removable storage container bounded by at least two sidewalls and having an open end for forming a storage cavity for receiving and for storing of phyto material extract, the removable storage container open end for engaging with the housing first end for forming a substantially enclosed heating chamber when the storage container is coupled with the housing where upon depressing of the switch, the slider assembly is slid within the housing and the conductive heating element is extended from the housing and heated to the predetermined vaporization temperature and contacts the phyto material extract for creating vapor emitted from conductive heating of the phyto material extract.

15. A vaporization device according to claim 14 wherein the heating element comprises a cylindrical heating element.

16. A vaporization device comprising:
providing a housing having a longitudinal axis and a housing end and a housing second end opposite the housing first end;
providing a slider assembly for sliding the longitudinal axis and in relation with the housing;
providing a vapor conduit extending axially between a first open conduit end and a second open conduit end, the vapor conduit having an outer conduit surface enclosing a fluid pathway extending between the first open conduit end and the second open conduit end, wherein the second open conduit end defining an inhalation aperture;
providing a heating element coupled with the slider assembly and proximate the housing first end;
heating of the heating element to a predetermined vaporization temperature wherein in use the predetermined vaporization temperature is sufficient to vaporize phyto material extract in contact with the heating element for providing phyto material extract vapor; and;
extending of the heating element away from the housing first end parallel with the longitudinal axis to contact phyto material extract contained within the storage cavity of the removable storage container;
inhaling from the inhalation aperture and creating a pressure gradient across the fluid pathway that draws ambient air from an ambient air input port from the external environment into the first open conduit end and the ambient air mixes with the phyto material extract vapor, and the mixed vapor and air are drawn through the fluid pathway to the inhalation aperture.

17. A vaporization device according to claim 16 comprising:
filling a removable storage container bounded by at least two sidewalls and having a storage cavity formed therein for containing phyto material extract;
releasably coupling of the removable storage container with the housing so the storage cavity faces the housing,
wherein extending of the heating element away from the housing first end parallel with the longitudinal axis comprises contacting phyto material extract contained within the storage cavity of the removable storage container.

18. A vaporization device according to claim 16 comprising:
providing a pivotable head coupled with the slider assembly proximate the second end of the housing and for extending past the housing second end, the pivotable head for being pivotable about a transverse axis that is approximately perpendicular to the coaxial longitudinal axis;
pivoting of the pivotable head being to a first orientation with the inhalation aperture oriented radially from the longitudinal axis;
sliding the pivotable head towards the housing second end along the longitudinal axis and extending of the heating element out from the housing first end;
filling with phyto material extract a removable storage container bounded by at least two sidewalls forming a storage cavity;
releasably coupling of the removable storage container with the housing so the storage cavity faces the housing,
wherein extending of the heating element away from the housing first end parallel with the longitudinal axis comprises contacting phyto material extract contained within the storage cavity of the removable storage container. and wherein extending of the heating element away from the housing first end parallel with the longitudinal axis comprises contacting phyto material extract contained within the storage cavity of the removable storage container.

19. A vaporization device according to claim 18 wherein the predetermined vaporization temperature is between 500 degrees Fahrenheit and 800 degrees Fahrenheit.

20. A vaporization device according to claim 16 comprising:
providing a pivotable head coupled with the slider assembly proximate the second end of the housing and for extending past the housing second end, the pivotable head for being pivotable about a transverse axis that is approximately perpendicular to the longitudinal axis;
rotating of the pivotable head about the transverse axis and extending of the heating element away from the housing first end parallel with the longitudinal axis.
